(19) United States
(12) Patent Application Publication
(10) Pub. No.: US 2025/0025022 A1
(43) Pub. Date: Jan. 23, 2025

(54) DUAL ROBOTIC ENDOSCOPE CONFIGURATION FOR TISSUE REMOVAL

(71) Applicant: Tamar Robotics LTD, Kibbutz Yagur (IL)

(72) Inventors: Noam HASSIDOV, Moshav Bustan HaGalil (IL); Hadas ZISO, Kiryat-Tivon (IL)

(73) Assignee: Tamar Robotics LTD, Kibbutz Yagur (IL)

(21) Appl. No.: 18/713,717

(22) PCT Filed: Nov. 29, 2022
(86) PCT No.: PCT/IL2022/051273

§ 371 Date: May 28, 2024

Related U.S. Application Data
(60) Provisional application No. 63/284,668, filed on Dec. 1, 2021.
(60) Provisional application No. 63/305,342, filed on Feb. 1, 2022.

(51) Int. Cl. A61B 1/00; A61B 1/018; A61B 17/00; A61B 17/32; A61B 34/30; A61M 25/01; A61M 25/06

(52) U.S. Cl. CPC A61B 1/00133; A61B 1/018; A61B 17/320016; A61B 34/30; A61B 2017/0034; A61B 2034/301; A61M 25/0113; A61M 2025/0681

(57) ABSTRACT

Dual surgical manipulator systems adapted for operating in stringently confined intrabody spaces, and/or for reaching such spaces via stringently confined access routes. In some embodiments, each of a pair of surgical manipulator arms introduced together and initially in parallel to each other is configured to approach a shared action zone from two different sides relative to their direction of introduction. Optionally, the arms may bend once to reach the shared action zone. One may bend by less than 90°, one may bend by more than 90°. One or more imagers may be provided which are configured to view the arms from a location lateral to their direction of introduction. The manipulator arms may be of a nested tubular design, which may contribute to reducing their complexity, and to their suitability for miniaturization.

### RELATED APPLICATIONS

This application claims the benefit of priority under 35 USC § 119 (e) of U.S. Provisional Patent Application No. 63/284,668 filed Dec. 1, 2021 and U.S. Provisional Patent Application No. 63/305,342 filed Feb. 1, 2022; the contents of which are incorporated herein by reference in their entirety.

This application is co-filed with PCT Patent Application entitled “ROBOTIC ENDOSCOPE CONFIGURATION FOR TISSUE REMOVAL”, having attorney docket number: 94831; its contents are incorporated herein by reference in their entirety.

### FIELD AND BACKGROUND OF THE INVENTION

The present invention, in some embodiments thereof, relates to the field of robotic and/or remote operated systems for use in minimally invasive surgical and diagnostic procedures, and more particularly, but not exclusively, to designs of surgical manipulators used in such systems.

A variety of surgeries have been demonstrated (and in some cases, are routinely performed) using robotic and/or remote operated systems. Minimally invasive surgical procedures in particular have a potential benefit from appropriately designed robotic and/or remote operated manipulators due to considerations, e.g., of size and/or flexibility. Remote-operated systems operate under the close guidance of motions by a surgeon-operator, optionally by direct mechanical linkage to those motions, or by translation of those motions to commands provided to one or more motors. Robotic systems may operate in part autonomously.

Endoscopic surgeries are used for a variety of interventions in a variety of organs and tissues. Surgical sites may accessed through natural orifices, and/or orifices constrained by location to a narrow channel. Surgical sites may be defined, at least in part, by internal lumenal spaces. Some lumenal spaces can be enlarged, e.g., by insufflation of gas, water, saline, blood, or CSF; others are limited, for example by a need or preference to avoid tissue damage. Surgical creation of lumens and orifices (e.g., incisions and/or evacuations) is common, but it is a goal of minimally invasive procedures to limit the severity of these interventions.

The contents of International Patent Publication Nos. WO2012/095845, WO2019/049154, and WO2020/208634 are included herein by reference, in their entirety.

### SUMMARY OF THE INVENTION

According to an aspect of some embodiments of the present disclosure, there is provided a surgical manipulator arm system comprising: an introducer, defining a lumen having a distal aperture; and first and second manipulator arms, sized to pass through and distally out of the introducer lumen, alongside each other and along a distal longitudinal axis of the introducer; wherein the first and second manipulator arms: each respectively define a lumen with an distal opening, the lumen and distal opening being sized to receive a surgical tool passing therethrough, deploy to orient their respective distal openings so that surgical tools exiting them advance towards a shared zone within which the respective surgical tool of each manipulator arm is operable; and wherein the advance is from mutually opposing directions along the distal longitudinal axis.

According to some embodiments of the present disclosure, each of the first and second manipulator arms, upon deployment distally from the introducer, comprises: a first straight section oriented along the distal longitudinal axis of the introducer, and reaching from the introducer to a curving section; the curving section, curved throughout its longitudinal extent to reach a deflection angle orienting the curving section's distal longitudinal axis oblique or perpendicular to the distal longitudinal axis of the introducer; and a second straight section, oriented along the distal longitudinal axis of the curving section, and reaching from a side of the curving section opposite the first straight section to the distal opening of the manipulator arm.

According to some embodiments of the present disclosure, the respective first straight sections of each of the deployed first and second manipulator arm are advanced from the introducer to different longitudinal extents, and the respective curving sections bent to different deflection angles.

According to some embodiments of the present disclosure, the deployed first manipulator arm is positioned with the deflection angle of its curved section being less than 90°; and the deployed second manipulator arm is positioned with the deflection angle of its curved section being 90° or more.

According to some embodiments of the present disclosure, the curved section bends through a radius of curvature which is less than a diameter of the lumen of the introducer.

According to some embodiments of the present disclosure, the deflection angle of the curved section adjusts according to how far the curved section is longitudinally advanced relative to the first straight section.

According to some embodiments of the present disclosure, the curved section bends unidirectionally between the first straight section and the second straight section.

According to some embodiments of the present disclosure, the curved section bends continuously throughout its extent.

According to some embodiments of the present disclosure, the radius of curvature is constant between the first straight section and the second straight section.

According to some embodiments of the present disclosure, the second straight section remains in a same plane with the first straight section as the curved section bends.

According to some embodiments of the present disclosure, a region of coplanarity extends from at least where the first straight section leaves the introducer, and through the curved section and second straight section up to the distal opening of the manipulator arm.

According to some embodiments of the present disclosure, the first straight section of the deployed second manipulator arm extends longitudinally beyond the distal end of the first straight section of the deployed first manipulator.

According to some embodiments of the present disclosure, the first straight section, the curving section, and the second straight section comprise tubular members, defining a lumen sized for passing a surgical tool out to and beyond the distal opening of the manipulator arm.

According to some embodiments of the present disclosure, at least a portion of the curving section deploys from inside the lumen of the first straight section, and at least a portion of the second straight section deploys from inside the curving section.

According to some embodiments of the present disclosure, the first straight section is controllable to rotate around a longitudinal axis of the first straight section, relative to the introducer.

According to some embodiments of the present disclosure, the second straight section is controllable to rotate around a longitudinal axis of the second straight section, relative to the curved section.

According to some embodiments of the present disclosure, the surgical manipulator comprises an auxiliary introducer inside the introducer, and wherein a lumen of the auxiliary introducer guides the relative positioning of the first and second manipulators as they deploy distally from the introducer.

According to some embodiments of the present disclosure, the lumen of the auxiliary introducer is sized so that the first and second manipulator arms are held in position by contact both with the lumenal wall of the lumen, and with each other.

According to some embodiments of the present disclosure, the auxiliary introducer is an endoscope comprising at least one camera positioned at a distal end of the auxiliary introducer.

According to some embodiments of the present disclosure, the manipulator arms deploy to define a shared action zone located beyond their respective distal openings, accessible by a tool deployed to extend straight beyond the distal opening of either of the manipulator arms.

According to some embodiments of the present disclosure, the shared action zone is positioned longitudinally distanced from the distal end of the first straight section of the first manipulator arm by less than a diameter-length of the introducer.

According to some embodiments of the present disclosure, the shared action zone is positioned radially distanced by at least one radius-length beyond a distal axial shadow of the introducer.

According to some embodiments of the present disclosure, the surgical manipulator arm system comprises a camera module, wherein the camera module deploys to orient a field of view of at least one camera of the camera module with a central view axis pointing proximally along the distal longitudinal axis.

According to some embodiments of the present disclosure, the surgical manipulator arm system comprises a second camera, oriented with a field of view having a central view axis pointing distally along the distal longitudinal axis.

According to some embodiments of the present disclosure, the surgical manipulator arm system comprises a side-pointing camera module, wherein a central viewing axis of a field of view of a camera of the camera module is oriented to point within 30° of an axis perpendicular to the distal longitudinal axis of the introducer.

According to some embodiments of the present disclosure, the central viewing axis of the camera oriented to point within 15° of the axis perpendicular to the distal longitudinal axis of the introducer.

According to some embodiments of the present disclosure, the camera deploys to a position radially beyond a distal axial shadow of the introducer.

According to an aspect of some embodiments of the present disclosure, there is provided a method of operating a surgical manipulator arm system, the method comprising: advancing first and second manipulator arms through and distally beyond an introducer, and along a distal longitudinal axis of the introducer; advancing the second manipulator arm distally further than the first manipulator arm; bending the first and second manipulator arms within a plane, orienting respective distal openings of each manipulator arm to opposing directions along the distal longitudinal axis; advancing a respective tool out the respective distal opening of each of the manipulator arms into a shared zone of action; and introducing a camera from the introducer and positioning it with a field of view including the shared zone of action.

According to some embodiments of the present disclosure, the method comprises bending the first manipulator arm by an angle of less than 90° away from the distal longitudinal axis.

According to some embodiments of the present disclosure, the method comprises bending the second manipulator arm by an angle of more than 90° away from the distal longitudinal axis, thereby orienting the terminus of the second manipulator arm to extend in a proximal direction along the distal longitudinal axis.

According to some embodiments of the present disclosure, each of the first and second manipulator arms is operated such that: the advancing advances a first straight section in an orientation along the distal longitudinal axis of the introducer, the bending bends a curving section to curve throughout its longitudinal extent to reach a deflection angle orienting the curving section's distal longitudinal axis oblique or perpendicular to the distal longitudinal axis of the introducer; and comprising advancing a second straight section from a side of the curving section opposite the first straight section, along the curving section's distal longitudinal axis.

According to some embodiments of the present disclosure, the method comprises bending the curved section through a radius of curvature which is less than a diameter of the lumen of the introducer.

According to some embodiments of the present disclosure, the bending comprises adjusting the deflection angle of the curved section according to how far the curved section is longitudinally advanced relative to the first straight section.

According to some embodiments of the present disclosure, the bending bends the curved section unidirectionally between the first straight section and the second straight section.

According to some embodiments of the present disclosure, the bending bends the curved section continuously throughout its extent.

According to some embodiments of the present disclosure, the radius of curvature is constant between the first straight section and the second straight section.

According to some embodiments of the present disclosure, the second straight section remains in a same plane with the first straight section as the curved section bends.

According to some embodiments of the present disclosure, a region of coplanarity extends from at least where the first straight section leaves the introducer, and through the curved section and second straight section up to a distal opening of the manipulator arm.

According to some embodiments of the present disclosure, the method comprises advancing the curving section from within the first straight section, and wherein advancing the second straight section advances it from within the curving section.

According to some embodiments of the present disclosure, the method comprises rotating the first straight section around a longitudinal axis of the first straight section, the rotating being relative to the introducer.

According to some embodiments of the present disclosure, the method comprises rotating the second straight section around a longitudinal axis of the second straight section, the rotating being relative to the curved section.

According to some embodiments of the present disclosure, the advancing is performed from within a lumen of an auxiliary introducer inside the introducer, which guides the relative positioning of the first and second manipulators as they deploy distally from the introducer; and wherein the lumen of the auxiliary introducer is sized so that the first and second manipulator arms are held in position by contact both with the lumenal wall of the lumen, and with each other.

According to some embodiments of the present disclosure, the shared zone of action is positioned longitudinally distanced from the distal end of the first straight section of the first manipulator arm by less than a diameter-length of the introducer.

According to some embodiments of the present disclosure, the shared zone of action is positioned radially distanced by at least one radius-length beyond a distal axial shadow of the introducer.

According to some embodiments of the present disclosure, positioning the camera comprises orienting the camera to have a field of view having a central view axis pointing proximally along the distal longitudinal axis.

According to some embodiments of the present disclosure, positioning the camera comprises orienting a central viewing axis of a field of view of the camera to point within 30° of an axis perpendicular to the distal longitudinal axis of the introducer.

According to some embodiments of the present disclosure, positioning the camera comprises orienting a central viewing axis of a field of view of the camera to point within 15° of an axis perpendicular to the distal longitudinal axis of the introducer.

According to some embodiments of the present disclosure, positioning the camera comprises deploying the camera to a position radially beyond a distal axial shadow of the introducer.

According to an aspect of some embodiments of the present disclosure, there is provided an endoscopic surgical system including: an introducer having a proximal end and a distal end; a steerable channel, including at least one tubular element sized to pass along the introducer between the proximal and distal ends; and a robotic motor controller within an enclosure, and configured to engage with the introducer and the steerable channel within the introducer, and including actuators configured to move at least the tubular element at least along a proximal-distal axis extending through the introducer; wherein the introducer engages with the enclosure in a region extending along a corner of the enclosure, such that sides of the enclosure extend at different angles away from opposite lateral sides of the introducer.

According to some embodiments of the present disclosure, the different angles meet at an angle of 120° or less.

According to some embodiments of the present disclosure, the introducer has a longer cross-sectional axis and a shorter cross-section axis, and engages with the enclosure in a relative orientation of the longer cross-section axis which is selectable among at least two different options.

According to an aspect of some embodiments of the present disclosure, there is provided an endoscopic surgical system including: an introducer having a proximal end and a distal end, with a cross-section having a longer cross-sectional axis and a shorter cross-section axis; at least two ports extending through the introducer between the proximal and distal ends, and arranged side by side along the longer cross-sectional axis; at least one steerable channel, including at least one tubular element sized to pass along one of the ports between the proximal and distal ends; and a robotic motor controller configured to engage with the introducer and the steerable channel within the introducer, and including actuators configured to move at least the tubular element at least along a proximal-distal axis extending through the introducer.

According to some embodiments of the present disclosure, the introducer is cross-sectionally sized to pass through human nostril into a nasal sinus.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an crasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable medium to receive instructions and/or data therefrom, process them, and/or store processing results in the same or another computer readable medium. The processing performed (optionally on the data) is specified by the instructions, with the effect that the processor operates according to the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIGS. 1A-1C schematically represent perspective, end-on and side views (respectively) of an introducer and endoscope of a manipulator system, according to some embodiments of the present disclosure;

FIGS. 2A-2C schematically represent perspective, end-on and side views (respectively) of introducer and endoscope of manipulator system, together with manipulator arms, according to some embodiments of the present disclosure;

FIGS. 3A-3C schematically represent perspective, end-on and side views (respectively) of an introducer and endoscope of a manipulator system including a distal camera module, according to some embodiments of the present disclosure;

FIG. 3D shows a distal-looking view of camera pod of a distal camera module, according to some embodiments of the present disclosure;

FIGS. 4A-4B demonstrate a 60°/120° bend configuration of a more proximally positioned manipulator and a more distally positioned manipulator in relation to a camera pod, according to some embodiments of the present disclosure;

FIGS. 4C-4D shows distal-looking and proximal-looking views from a camera of a camera pod (FIG. 4C) and a camera of an endoscope (FIG. 4D), according to some embodiments of the present disclosure;

FIGS. 4E-4G schematically represents a manipulator system comprising a retractor scaffold, according to some embodiments of the present disclosure;

FIG. 4H schematically represents a manipulator system comprising an ultrasound scanning probe, according to some embodiments of the present disclosure;

FIG. 5A-5E show views of a manipulator system comprising a camera module configured to view manipulator arms from a lateral position, according to some embodiments of the present disclosure;

FIG. 5F shows a view from a camera of a camera pod, according to some embodiments of the present disclosure;

FIGS. 6A-6G show views of a manipulator system comprising a distally-mounted camera module, configured to bend around proximally to provide a lateral view of manipulator arms, according to some embodiments of the present disclosure;

FIG. 6H shows a view from a camera of a camera module, according to some embodiments of the present disclosure;

FIGS. 7A-7N illustrate a variety of positioning configurations of manipulator arms, according to some embodiments of the present disclosure;

FIGS. 8A-8D schematically illustrate how relationships between camera position and arm position may be adjusted to maintain a suitable working distance between camera(s) and an operational region of the arms, according to some embodiments of the present disclosure;

FIG. 9A is a schematic flowchart of a method of positioning manipulator arms, according to some embodiments of the present disclosure;

FIG. 9B is a schematic flowchart of a method of positioning manipulator arms, according to some embodiments of the present disclosure;

FIG. 10 schematically illustrates degrees of freedom of a pair of manipulator arms, according to some embodiments of the present disclosure;

FIGS. 11A-11C schematically illustrate the extent of a distal axial shadow of an introducer, according to some embodiments of the present disclosure;

FIGS. 12A-12C schematically represent a configuration of a modular robotic endoscope system, according to some embodiments of the present disclosure;

FIGS. 13A-13B schematically represent an expanded configuration of a modular robotic endoscope system, according to some embodiments of the present disclosure;

FIG. 13C schematically represents an alternative expanded configuration of a modular robotic endoscope system, according to some embodiments of the present disclosure;

FIGS. 14A-14B schematically represent an expanded configuration of a modular robotic endoscope system, according to some embodiments of the present disclosure;

FIGS. 15A-15B schematically represent a-port modular robotic endoscope system, according to some embodiments of the present disclosure; and

FIG. 15C schematically represents a port arrangement of a-port modular robotic endoscope system, according to some embodiments of the present disclosure.

### DESCRIPTION OF SPECIFIC EMBODIMENTS OF THE INVENTION

The present invention, in some embodiments thereof, relates to the field of robotic and/or remote operated systems for use in minimally invasive surgical and diagnostic procedures, and more particularly, but not exclusively, to designs of surgical manipulators used in such systems.

### Overview

A broad aspect of some embodiments of the present disclosure relates to dual surgical manipulator systems adapted for operating in stringently confined intrabody spaces, and/or for reaching such spaces via stringently confined access routes.

Certain types of surgical manipulations benefit from (and may practically require) manipulation of tissue and/or surgical apparatus such as suture via a plurality of members simultaneously present at the site of surgical intervention. Benefit may be in part inherent to the operation being performed; for example, one tool may grip tissue to keep it in place, exposed, and/or relieved of tension, while another tool is operated to cut, penetrate, and/or fasten. Since they provide a manipulator per hand of a single human operator, dual manipulator configurations also have potential advantages as an “imitation pair of hands”.

Tools may be considered as components separate from the manipulator arms themselves, although they are optionally provided together. In surgical manipulators designed for use in endoscopic surgery, an arm may be designed with one or more lumens (each referred to herein as a working channel). A surgeon-selected tool may be advanced to the distal terminus of this lumen for use, and optionally withdrawn and exchanged for another tool during a procedure. Working channels vary considerably in width depending on surgical application, for example, from about 1.2 mm to about 12 mm (e.g., 2.8 mm). Maximum outer diameters of tools designed for use with these lumens vary in size too. For smaller tool outer diameters, there may be design constraints on the tool's own degrees of freedom and/or strength. However, smaller lumenal diameters may in turn be taken advantage of to allow providing a smaller overall introducer size.

Apart from introducer size, there is another set of potential size constraints on surgical manipulator designs, which is the size and shape of the working volume of and around the surgical field. This working volume may be constrained, for example, by the size and/or natural elasticity of a natural cavity. The size of the working volume may limited, for example, by a need or desire to minimize trauma, e.g., to avoid unnecessary cutting of bone or neural tissue, and/or damage to membranous structures.

“Intuitiveness” is a potential advantage for a dual manipulator system, e.g., insofar as intuitiveness may simplify operator training, reduce cognitive load, and/or improve transferability of skills. Intuitiveness may be analyzed as comprising a number of factors, however, and an instrument design may provide and/or emphasize only some of these, perhaps constrained by other considerations such as targeted overall introducer size and targeted working volume size and/or shape. Thus, there is in general a problem of how to provide, for a given set of size constraints, an acceptable and/or potentially improved level of control and intuitive operation for a surgical manipulator.

Among aspects of intuitive operation applicable to the design of a dual manipulator system are (1) visualization of the workspace and the being performed within it, (2) preventing the two manipulator arms from interfering with each other's operation, and (3) making it possible to bring the manipulator arms together in a way that allows them to assist each other. In particular, there is a potential advantage in a surgical manipulator system having the capability to bring two manipulator arms together within a shared action zone located beyond their respective lumens, which said zone can be visualized by a camera of the system while the two manipulator arms work co-operatively.

Where it may not otherwise be clear that tools extending from the manipulator lumens can operate or are operating co-operatively, a “shared action zone” may be identified to exist according to a geometric method. First, suppose a midpoint of the shortest line connecting the distal-ward extensions of the central longitudinal axis of each of the two manipulator arms. The shared action zone exists if this midpoint is, for each of the two manipulator arms, within the right cylindrical volume along an axial extension having three times the radius of the lumenal cross-section of the manipulator, and within the reasonable extent to which tools may be advanced from these lumens and remain useable. In the absence of reference to a particular tool's capabilities, a selected distance (for example of 1-3 diameters of the lumen) may be used as this distance. In unusual cases, the tool may be advanced by a larger amount from a manipulator arm to reach a position where it operates co-operatively with a tool of the other manipulator arm. For example, it may be advanced up to 12 diameters of the lumen. Such “long reach” cases may occur where the tool shaft itself is relatively stiff (and self-supporting) compared to the loads it experiences and/or the required precision of its positioning; and/or where the working volume is so restricted that the reduced diameter of the tool provides a meaningful advantage (e.g., in visualization and/or maneuverability) compared to the somewhat larger diameter of the manipulator arm.

The shared action zone may in any case be considered to be centered on the above-mentioned midpoint, e.g., for purposes of resolving direction of approach. The descriptions herein do not rely on defining the spatial extent of the shared action zone.

Functionally, and whether or not the geometrical definition applies, a shared action zone may be understood to exist, for example, when two tools are in a position from which one or the other of them can undergo a simple movement (e.g., a movement comprising operation of just one degree of freedom of movement) to contact the other. This does not preclude cases where two or more degrees of freedom are used to produce contact. Co-operation does not necessarily require contact, cither; for example, co-operative operation may comprising one tool cutting into a tissue region which is stretched, braced, or otherwise held in a deformed position by the other tool.

Approaching a shared action zone from at least partially opposing sides provides potential advantages for keeping tools (and the manipulator arms that introduce them) out of each other's way. Mutual approach from a partially opposing angle is also potentially advantageous for the types of co-operative motions the two tools can perform—it allows the tools push toward and pull away from each other (shearing motions are also potentially available). A difference in approach angle to the shared action zone of, for example, 30° or more may suffice to gain this advantage. In some embodiments of the present disclosure, the difference in approach angle to the shared action zone is at least 60°, or at least 90°. Herein, this angle is referred to as the inter-manipulator angle. Since axial extensions from the manipulator arms distal ends in a given position do not necessarily meet, this definition of an inter-manipulator angle may be understood as a difference in their angle coordinates relative to a common reference.

The angle of visualization (e.g., from a light-imaging camera) on to the shared action zone preferably is selected so that the shared action zone is minimally obscured by either manipulator (although the tools themselves within the shared action zone may by necessity obscure some portion of it). Moreover, there is a potential advantage for intuitive operation if this visualization angle is selected such that the shared action zone is viewed with one manipulator on the left, and the other on the right (similar to a natural view from the head of hands brought together in front of the body).

A camera position looking on to the shared action zone and located near (e.g., within 15° of or within 30° of) a plane making a same angle with the axial extension of each manipulator can be suitable to reduce manipulator viewing interference, as well as suitable to provide a view roughly equivalent (but mirror-imaged) for monitoring each manipulator. The camera's field of view can be rotated so that this plane extends from top to bottom, placing one manipulator on the left, and one on the right. In some embodiments, cameras are arranged in a pair; e.g., on either side of the plane, or otherwise positioned so as to provide a stereoscopic view of the shared action zone. Stereoscopic use and/or position is not required, however; in some embodiments, the provision of a plurality of cameras is simply used to allow different views, either of which may be preferred, depending on a current position of the manipulator arms and/or tools used with them. For example, a manipulator arm may block the view from one camera onto a region of interest, but not the other.

Potentially interfering with achieving the above-described geometrical arrangement of camera viewpoint(s) and manipulator arms: it is preferable in many surgical situations (e.g., endoscopic surgeries) to introduce manipulator arms to the surgical field through a single small aperture—an incision, or a natural body orifice. Moreover, the manipulator arms commonly share a single introducer, constraining them to enter the surgical field on close (e.g., adjacent) parallel trajectories (their “introductory trajectories”). Camera introductory trajectories may also be from along a close parallel trajectory to those of the manipulator arms. Use of a rigid introducer may be indicated for particular uses cases, e.g., to help ensure stability, predictability, and/or reproducibility of positioning.

Available space may be constraining in some embodiments of the present disclosure, and this potentially affects camera placement. In particular, arm diameter may be decreased (e.g., to about 4-5 mm) to the extent that it is around the same diameter as a usefully large camera lens assembly. It is a potential advantage to avoid having to expand the introducer so that its cross-sectional area can include, e.g., at least two such arms and at least one camera lens. Accordingly, in some embodiments of the present disclosure, one or more cameras are placed on a distal extension of the endoscope assembly, and linked back to the rest of the endoscope assembly, e.g., through an aperture smaller than the camera lens itself. A camera may be positioned, for example, with a side-looking field of view or a proximal-looking field of view. An at least partially distally-looking field is optionally provided, e.g., by advancing the lens and associated camera only a small distance distally, but also angled relative to the proximal-distal axis so that its central viewing angle includes both a distal-looking component, and a lateral-looking component.

Being parallel and adjacent (or nearly so) at their point of introduction, additional design measures are used, in some embodiments, to allow achieving a suitably large inter-manipulator angle at the shared action zone. For example, each single surgical manipulator of the pair may be provided with a plurality of joints that bend in opposing directions. A proximally positioned joint, for example, bends to orient the manipulator arms to point first away from their introductory trajectories (and away from the intended shared action zone). Moving distally along each manipulator, this creates the distance that allows a bend in a more distal joint to re-orient each manipulator's distal axis extension back toward the shared action zone. This creates the targeted inter-manipulator angle (e.g., of at least) 30°. In this commonly used (and typically symmetric) configuration, the camera can be oriented to view along its original introductory trajectory (which is often also its original orientation). Insofar as this configuration also moves more proximal manipulator portions to the sides of the camera field of view, this configuration may also help provide clear viewing of the shared action zone.

It has been mentioned above, however, that the size and shape of the working volume of (and around) the surgical field may constrain what movements may be allowable for a surgical manipulator (in a given targeted use). To be available, the two-jointed solution just described requires sufficient “elbow room”-lateral space allowing the manipulator arms to travel sufficiently far away from the introductory trajectory of the manipulator arms that they can eventually bend back to meet with a comfortably large inter-manipulator angle. It should be emphasized that this lateral space is not required for access to the surgical field as such; the need for it arises from other working constraints.

An aspect of some embodiments of the present disclosure relates to systems comprising a pair of surgical manipulator arms, each with about the same direction of introductory trajectory, wherein said manipulator arms approach a shared action zone from two different sides relative to that direction of introductory trajectory. For example, a first manipulator bends by less than 90° from its introductory trajectory to reach a targeted shared action zone, while a second manipulator extends distally beyond the targeted shared action zone, then bends by more than 90° to reach proximally back toward the shared action zone.

In some embodiments, all bends of a respective surgical manipulator arm are in the same direction of curvature. The curvature of a manipulator arm may all be co-planar; or if not entirely co-planar, the direction of curvature is constant as projected onto a planar surface along which the projected extent of the manipulator is the longest.

In some embodiments, bending of each respective manipulator occurs continuously along a single respective region of the manipulator; optionally with a constant radius of curvature. The bend of this continuous region begins, in some embodiments, from a position within the “distal axial shadow” of an introducer used to introduce the manipulator arms into a surgical field. The bend may complete within the distal axial shadow, or it may complete beyond it. Optionally, an outer radius of curvature of the bending is less than the diameter of the introducer.

Herein, should the extent of positions within the distal axial shadow of the introducer be otherwise unclear: a volume “within the distal axial shadow” of a longitudinally elongated element is a volume surrounding an axis of the element that extends longitudinally from and distal to the element; the volume having also the distal cross-section of the element, aligned to the axis in the same way as the distal cross-section. In the case of a final distal taper and/or bevel of the element, the most distal cross-section before this taper/bevel is used.

It is noted that since it is sized to the introducer cross-section, the volume of the distal axial shadow needs to be clear (and potentially made clear by operations such as tissue removal) in order to advance the introducer further. Moreover, and particularly if tissue clearing operations into solid tissue are limited to within the distal axial shadow, then the advancing introducer may serve as a support which prevents and/or reverses tissue collapse. Furthermore, elements extending from near the circumferential periphery of the lumen of introducer are also well-positioned to block movement of tissue, e.g., limit it to about the wall-thickness of the introducer. As a result, confining at least an initial phase of operations to remove (excavate) tissue to within the distal axial shadow has potential advantages for controlling (e.g., substantially preventing) tissue movements into new locations which may make the current position and/or shape of a tissue targeted for treatment unclear.

In some embodiments of the present disclosure, the shared action zone of two arms is optionally positionable either within or outside of the distal axial shadow. When outside of the distal axial shadow (for example, after excavations begin outside of the distal axial shadow), the region of the shared action zone may be maintained clear of obstructions by the positioning of one or more supporting elements also deployed from the introducer.

The other supporting elements may comprise, for example: a distally positioned element which supports tissue on a distal side of the available working volume, a laterally positioned element which also extends distally and blocks tissue ingress into the working area of the distal axial shadow, and/or a laterally positioned element which is expandable (laterally) to positions outside the distal axial shadow.

The expandable element in particular (also referred to herein as a scaffold or retractor scaffold) may be expanded in a controlled fashion as tissue removal proceeds so that it maintains tissue laterally beyond and in contact with it substantially in its original position. A portion of adjacent unsupported tissue may be removed, and the scaffold moved underneath it and expanded as necessary. It should be noted that with suitable care, the scaffold can also be used to restore partially collapsed or otherwise moving tissue to its original location, so long as the original position of the introducer remains constant or otherwise well-defined (e.g., limited in its degrees of freedom, known by imaging, known by control history and/or status, and/or by other sensing). For example, if the scaffold is rotated away from a certain side of an excavated region, that region may be freed to move (e.g., partially collapse). However, when the scaffold returns to the same position, it will contact the same tissue, with the effect of restoring its position to about the same as it was previously.

In some embodiments, with the shapes of the two surgical manipulator arms each respectively bent to reach a shared action zone, a center of the shared action zone itself is located laterally from an introducer used to introduce the surgical manipulator arms; that is, outside of its distal axial shadow. The lateral displacement of this center may be at least half the distance of its displacement along the longitudinal axis of the introducer's axial distal axial shadow. For example, the lateral displacement may be at least 50%, at least 75%, or at least 100% of longitudinal axis displacement.

As an example, a proximal-approach manipulator may bend 60° away from the longitudinal axis, optionally initiating this bend immediately upon exiting the introducer. A second, distal-approach manipulator may bend 120° away from the longitudinal axis, initiating this bend beyond the longitudinal displacement of a target region (e.g., the shared action zone). The bend may begin, for example, at about double the longitudinal displacement of the target region, optionally minus allowance for the manipulator's own radius, and for its radius of curvature. The center of the shared action zone in this case will be laterally displaced by roughly the height of the triangle thus defined (a triangle wherein two sides are defined by sections of the second manipulator, and the third by the first manipulator after its bend). Before taking into account allowances for radius of curvature and element radius, this results in a lateral displacement about 1.7 times larger than the longitudinal displacement. Alternatively, with bends of 45° and 135°, the ratio will be about 1. Or, with less extreme bending of the second manipulator (and greater bending of the first manipulator), the ratio will tend to increase.

When the manipulators are introduced side-by-side and bend in different planes, there may be additional rotation of one or both of the manipulator arms (e.g., about a distal longitudinal axis of the introducer) in order to align their distal ends so that each points at the center of the shared action zone. For example, one manipulator arm may rotate by 5°-10° toward the other. However, the manipulator arms are not necessarily oriented toward a same point. They can share a working zone, for example, when the distal longitudinal axis of one manipulator arm is offset to allow cutting at tissue location away from a location where the other manipulator is gripping tissue to stretch or otherwise manipulate it.

In some embodiments, the first bend of a manipulator (e.g., a manipulator which bends more than 90° to reach a target region) occurs within the distal axial shadow of its introducer, and after a stiff section which is longer than a longitudinal displacement of the target region along the longitudinal axis.

Regarding construction of manipulator arms having positioning properties as described above: in some embodiments, the manipulator arms are constructed of nested tubular elements. The tubular elements may be flexible in part, (for example in proximal portions, under sufficient load, and/or along sufficient length). However, at the working end of the manipulator, the tubular elements have stiffness properties selected according to their particular function in the manipulator.

Each manipulator, in some embodiments, is constructed with at least three layers that surround some longitudinal portion of the lumen of the manipulator's working channel. The layers can be moved longitudinally relative to each other. In overview, the outer and inner layers define stiff segments of the manipulator, and the middle layer defines a bend which connects the two segments. At least the segments can each rotate, and both the bend layer and the segment layers can be extended and retracted. Extending and retracting the bend layer changes the degrees of bend articulation. Extending and retracting the segment layers changes the segment lengths. Together, this provides several degrees of freedom per manipulator (e.g., five degrees of freedom), which may be used to position endoscopic tools in a target area to perform tissue manipulations and other tasks such as suction, irrigation, or illumination.

In more detail, an outer layer may be the stiffest of the three layers, and sufficiently stiff that it extends straight along its own distal longitudinal axis as it is advanced from an introducer.

A middle layer includes a particular flexible portion near its distal end (e.g., up to the end, or within up to 1-10 mm of the end). The flexible portion converts between two configurations: (1) a straightened configuration while confined within the outer layer, and (2) a curved configuration upon advancing distally beyond the outer layer and out of confinement. Total curvature may be reached gradually, e.g., proportional to the extent that the flexible portion is advanced beyond the confinement of the outer layer.

The curved configuration upon advance beyond confinement may be caused, for example, because the middle layer is elastically predisposed to curve when unconfined; e.g., made of a superelastic nitinol allow with a shape set to assume such a curve. Additionally or alternatively, curvature may be induced by magnetic attraction between different segments of the middle layer, by tension exerted through a longitudinally extended member extending proximally from a distal connection with the middle layer, or by another means. The nitinol construction in particular has the potential advantage of requiring no extra use of manipulator volume for control elements.

Straight or curved, the middle layer retains substantially a constant lumenal cross section in both its main configurations. In some embodiments, this is accomplished by segmenting and/or slitting the middle layer along its flexible portion. For example, a nitinol tube may be slotted, and set to elastically assume the curved configuration with slot edges on an inner curvature side pressed toward each other (optionally touching), and slot edges on an outer curvature side separated away from each other. The radius of curvature may be set by the slot gap sizes and shapes. The flexible portion is optionally set to continue to elastically exert curving force even after the slot edges begin to interfere with each other, which may help to lock the curvature in place against, self-straightening forces exerted by the inner layer, described herein below.

Upon fully advancing from the outer layer, the curvature of the flexible portion of the middle layer may extend through a range of up to 180°, in some embodiments. The maximum range of curvature may be even somewhat larger; for example, enough to allow the manipulator to bend back enough that its distal end contacts the manipulator itself at a more proximal location. It should be understood that the maximum curvature may be less depending on how the particular manipulator is to be used, and that the maximum curvature is not necessarily the same for both manipulator arms of a pair. For example, a first manipulator which is to be longitudinally advanced less than a second manipulator may have a maximum range of curvature of 90° or less, while the second manipulator has a maximum range of curvature beyond 90°.

The inner layer, in some embodiments, comprises a tubular element which is flexible enough to follow the bending of the middle layer, but also stiff enough that once past that bend, it extends stiffly and straight beyond the bend, including remaining straight where it advances distally beyond the middle layer altogether. The greater the force holding the curvature of the middle layer's flexible region in place, the stiffer the inner element can be. To maintain straightness, the inner element also benefits from being operated with tool forces that concentrate along the distal longitudinal axis of the inner element, and exert relatively little lateral deflection force.

The layered construction is an example. In some embodiments, a manipulator arm more generally comprises two straight segments configured with a prismatic or cylindrical joint (longitudinally sliding or longitudinally sliding and circumferentially rotating), the two straight segments being separated by a curved section which acts as a revolute joint between them.

Various configurations of manipulator providing at least some of the just-described operational properties are described, for example, in International Patent Publication Nos. WO2012/095845, WO2019/049154, and/or WO2020/208634, the contents of which are included herein by reference, in their entirety. It should be noted that the manipulator arms just described are suitable for direct manipulation by hand-operated linkages. This provides potential advantages for providing force feedback to the hands of the operator, inherently generated by forces experienced by the manipulator. However, it is not excluded that the manipulator arms are operated by a motorized controller. Optionally, combination of human and machine movements is supported; e.g., machine-produced corrections superimposed on human-instigated movements (for example, to smoothly reach a target), and/or human supervision of machine-produced movements to halt or modify them, e.g., in case the human notices a condition that the machine is not configured to respond to. The controller may receive movement commands from any suitable human-operated input device, and translate the inputs into the movements of the manipulator elements which correctly correspond to the human input. Additionally or alternatively, commands may originate as automatically generated commands, for example, commands automatically generated so as to reach a selected target or position in space. Tools inserted to the manipulator arms may likewise be operated manually or by an intervening motor controller; and in the latter case also with commands generated optionally from user inputs, or automatically.

An aspect of some embodiments of the present disclosure relates to systems comprising a pair of surgical manipulator arms, and providing a camera for viewing those manipulator arms which is oriented with its a central viewing axis substantially perpendicular to a longitudinal axis defined by an introductory trajectory of the manipulator arms as they enter the surgical field from an introducer. In some embodiments, this arrangement places one of the manipulator arms mainly to one side (e.g., the left side) of the central viewing axis, and the other manipulator mainly to the opposite side. More particularly, the manipulator arms may be positioned to define a shared action zone, with one manipulator longitudinally advanced distally but angled proximally, and the other manipulator less distally advance and angle distally.

In some embodiments a plurality of cameras are provided, positioned, for example, to provide different viewing angles onto the surgical manipulator arms and/or their shared action zone. Optionally, the cameras are used stereoscopically. In this case, the central viewing axis may be considered as the axis extending from the midpoint between the two cameras.

In some embodiments, at least one camera advances on a carrier distally out of an introducer with its central viewing axis already oriented in a lateral-looking direction, e.g., with a field of view such that “distal” (of the system of manipulator arms) is on the left, and “proximal” is on the right. This example is without excluding that the mapping of distal/proximal could be respectively to image top and bottom, or inverted from either of these options. The shared action zone is established by maneuvering the manipulator arms so that one enters the field of view from the proximal side of the field of view, while the other advances across the proximal-to-distal direction (either in view, or outside of the field of view), curves on a joint, and extends back from the distal side of the field of view toward its proximal side. The shared action zone may be positioned somewhat away from the camera position in depth by the angled lengths of the distal segments of the two manipulator arms. Optionally, the camera is further distanced from the action zone by movement of its carrier; e.g., the carrier may be jointed or curved.

Alternatively, in some embodiments, the camera is re-oriented by a process of deployment (e.g., deployment of its carrier) to reach its vantage onto the two manipulator arms and/or their shared action zone. For example, in some embodiments, at least one camera is located on a carrier which advances distally out of an introducer, and then itself bends back around through about 180°. The diameter of the bend causes the camera to be thereby translated to a lateral position somewhat distanced from the manipulator arms. In this configuration, the distal segments of the manipulator arms may be oriented so that they occupy opposite sides of the camera's field of view (e.g., with a central view axis extending through a region between the distal termini of the manipulator arms). A roughly-side on view is potentially advantageous for reducing foreshortening (e.g., by making both arms appear at about the same size in the camera view). However, angles deviating from a side-on arrangement (e.g., by ±45° may give a sufficiently clear view of the work being performed in a shared action zone distal to each of two manipulator arms. The camera orientation may furthermore be elevated (e.g., by about 30°-45°) so that the central view axis is oriented more toward the center of a shared action zone of the two manipulator arms.

In another example, a camera on a carrier (e.g., a carrier comprising its umbilical connection to power and/or data transmission lines) may be initially oriented to point distally (backward) along a longitudinal axis (e.g., along its trajectory of introduction), with the carrier being deployable from an introducer to bend outward by about 90° (e.g., as it advances). As a result, the camera again ends up pointing laterally across the longitudinal axis, and toward the manipulator arms and/or their shared action zone. The camera angle need not be transformed through a full 90°. It may be initially and/or finally oblique in orientation compared to the distal longitudinal axis of the introducer, for example.

While lateral displacement of the camera also results in the use of more lateral space, this may be acceptable in some situations where the space limitations are primarily restrictive to the introduction of the manipulator arms (e.g., via a narrow passageway such as a ureter), and less restrictive once the manipulator arms reach their intended target (e.g., a relatively large lumen such as the interior of a bladder).

An aspect of some embodiments of the present disclosure relates to modular designs of robotically controlled endoscopic devices. In some embodiments, an introducer for a robotic arm device couples to at least one a robotic controller in order to provide robotic control to one or more arms which pass through the introducer.

The introducer optionally includes a plurality of ports sized to allow the arms to pass. Optionally, at least one of the ports is used for an endoscopic device comprising an imager.

In some embodiments, the introducer is straight and rigid. In some embodiments, each of a plurality of robotic controllers couples to a respective port of the straight and rigid introducer, each at or near an edge and/or corner of its respective enclosure. The enclosures are thereby clustered around the introducer, for example radially arranged.

In some embodiments, each of a plurality of robotic controllers couples to a respective straight and rigid introducer, each at or near an edge and/or corner of its respective enclosure. The straight and rigid introducers are optionally aligned adjacent and parallel to each other, so that the enclosures are thereby clustered around the introducer; for example radially arranged. Optionally, the introducers are positioned with more independence in orientation, i.e., converging distally to a common working area from more widely separated positions proximally.

In some embodiments, the introducers are sized and shaped to pass through a human nostril. Nostrils are commonly oblong in shape, e.g., with a minimum cross-section about 10 mm along a long axis, and about 5 mm along a short axis. There are two of them; and although divided by a septum distally, they lead to a common volume within the sinuses. In some embodiments, introducers are provided comprising a plurality of ports arranged within an oblong-cross section; e.g., two circular ports enclosed within a rectangular cross-section with rounded ends. The overall cross-section may fit within a rectangle, e.g., about 10 mm by 5 mm in dimension. Optionally, the introducers are used together in a pair, each with its own robotic controller for controlling one or more steerable channels (manipulator arms) which pass through the port(s) of the introducer. Optionally, at least one port of at least one of the introducers is occupied by an endoscope, e.g., a device providing a distally mounted camera and illumination devices. Because the introducers are independent, one of them may be withdrawn at any time, optionally to be replaced with another tool, e.g., a flexible endoscope or other device.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### Surgical Manipulator Arm System Configurations

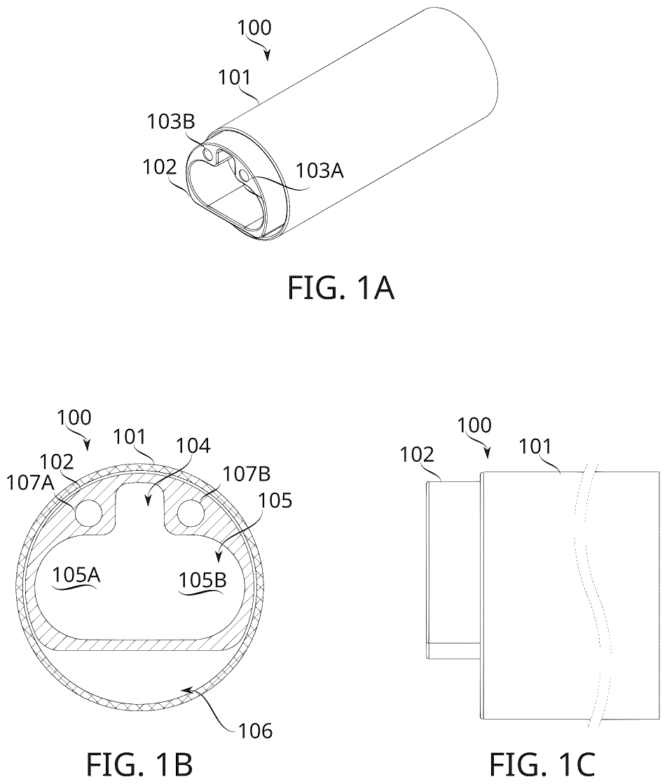

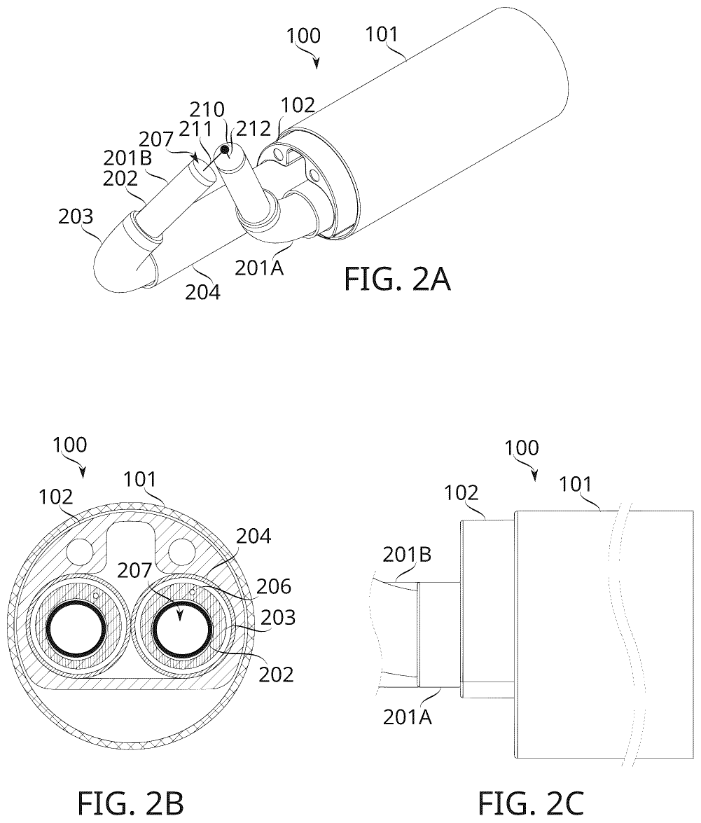

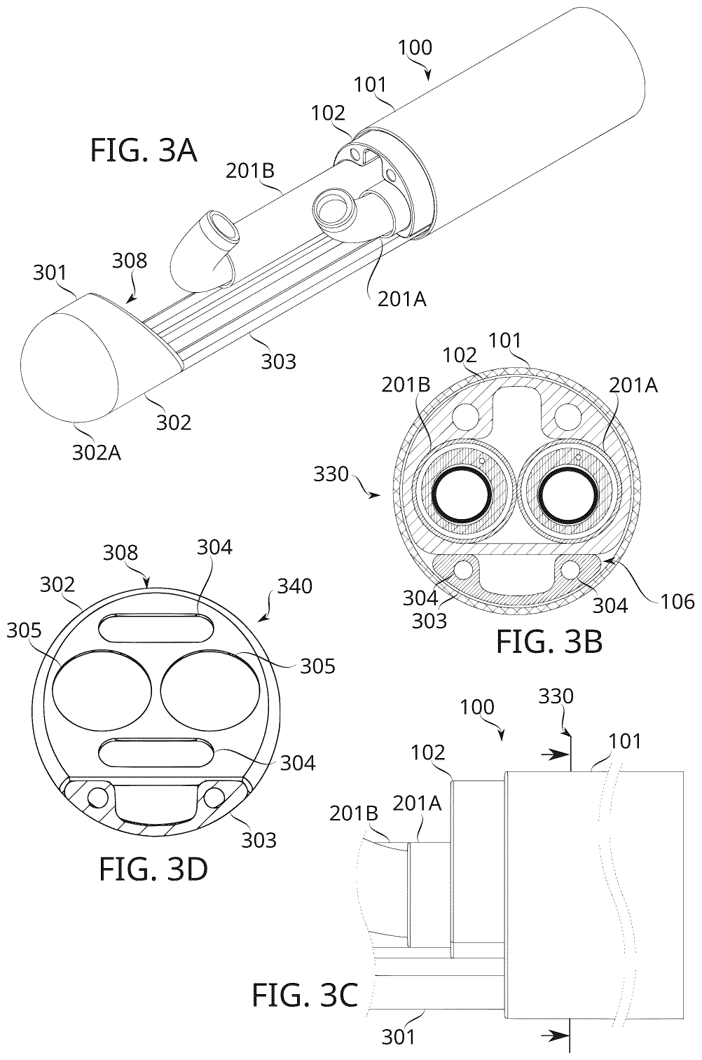

Reference is now made to FIGS. 1A-1C, which schematically represent perspective, end-on and side views (respectively) of an introducer 101 and endoscope 102 of a manipulator system 100, according to some embodiments of the present disclosure. These three figures are part of a sequence of figures continuing with FIGS. 2A-2C, showing stages in the deployment of manipulator arms with system 100. FIGS. 3A-3C, 4A-4C, 5A-5C, and 6A-6E build on the basic manipulator configuration of Figures IA-2C with different optional camera configurations. The longitudinal position of the cross-section of FIG. 1B is just before the distal terminus of introducer 101.

In the example shown, introducer 100 may simply be a hollow tube, with an overall outer diameter of, for example, about 10 mm. The outer diameter may be considered as larger or smaller than 10 mm in some embodiments, with other dimensions given in the following descriptions scaled proportionally. A limitation of the minimum size of the device may arise from the mechanical properties of the manipulator arms (which, below a certain wall thickness, may become too flimsy), and/or from the size of a working channel lumen required to advance desired standard tooling to the site of surgical operations. Optionally, larger sizes may be readily constructed, but their use may be constrained by practical considerations such as the available room to maneuver instruments.

Endoscope 102 illustrates a “low volume” design, in which only manipulator arms extend distally into the working volume, unless supplemented by additional, optional, tools. The cameras 103A, 103B are placed on a distal face of endoscope 10, facing distally (lumens 107A, 107B may be used as passages through which the camera is interconnected with equipment on a proximal side of the introducer 101). While this provides a somewhat unusual view of the manipulator arms and their shared action zone, elimination of extra space requirements may make this compromise worthwhile in some scenarios. In any case, one or more additional cameras are optionally introduced through working channels of endoscope 102, for example as next described.

In some embodiments, a working channel 105 of endoscope 102 is sized for the side-by-side passage of two manipulator arms, each having an outer diameter in the range, for example, of about 4-4.5 mm. For example, one manipulator may occupy generally lobe 105A of the lumen, and another may occupy lobe 105B. In the example shown, there is also a third lobe 104 of lumen 105, and furthermore a separate working channel 106 formed in a space left open between endoscope 102 and introducer 101. These channels are optionally used to introduce irrigation and/or suction, or to introduce further tools, which optionally may include one or more cameras which are positionable somewhere distal to the distal face of endoscope 102.

With particular reference to the shape of lumen 105, the lobed structure allows optionally passing individual devices which temporarily share some regions of the overall lumenal cross section. For example, a first-advanced device may comprise an umbilical which is sized to fit within lobe 104, and a head which uses more space, for example, also including both regions 105A and 105B. The head can be advanced beyond the distal terminus of endoscope 102, freeing regions 105A-105B for other uses. A second-advanced device may have an umbilical occupying lobe 105A (for example), but a head occupying both regions 105A and 105B. After its own head is advanced sufficiently, a third-advanced device may be advanced distally to and optionally past the distal terminus of endoscope 102 through lobe 105B. It may be noted also that in the side-by-side advance of two manipulator arms through lumen 105 (via lobes 105A, 105B, respectively, e.g., as shown in FIGS. 2A-2C), each manipulator acts as a bearing on the other on one side, keeping each other in place within the overall confines of lumen 105. Lobe 104 is sized with an opening small enough that manipulator arms of sufficient size are prevented from slipping into it. These features of shape and mutual interactions may help maintain the structure of the relative positioning of elements as they advance to the distal terminus of endoscope 102, even though they may otherwise be introduced “loosely” relative to one another.

Reference is now made to FIGS. 2A-2C, which schematically represent perspective, end-on and side views (respectively) of introducer 101 and endoscope 102 of manipulator system 100, together with manipulator arms 201A, 201B, according to some embodiments of the present disclosure. The longitudinal position of the cross-section of FIG. 2B is just before the distal terminus of introducer 101.

Each of manipulator arms 201A, 201B is constructed as an at least three-layer device, comprising outer layer 204, middle layer 203, and inner layer 202. The three layers concentrically surround central lumen 207.

Outer layer 204 and inner layer 202 each define respective straight segments of a manipulator. The straight segment defined by outer layer 204 extends straight out of lumen 105, along the distal longitudinal axis of lumen 105. Inner layer 202 defines a straight segment which extends distally from middle layer 203.

Middle layer 203 introduces a curvature between these two segments. In the example shown, an extended longitudinal member 206 attaches near a distal end of middle layer 203. Tension on member 206 causes middle layer 203 to assume a curved configuration as it is released from confinement within outer layer 204 and/or lumen 105. Inner layer 202 passes around this curvature, so that the more distal segment defined by inner layer 202 is directed away from the distal longitudinal axis of lumen 105. Inner layer 202 tends to self-straighten when not confined to a curved shape, so that it extends straight where it is distal from middle layer 203.

Of particular note is the relative configuration of the manipulator arms 201A, 201B shown in FIG. 2A. For manipulator 201A, the portion of outer layer 204 advanced beyond endoscope 102 is quite short, so that the curvature of middle layer 203 begins almost immediately beyond the end of endoscope 102. The curvature is approximately 60°. Inner layer 202 is advanced far enough that it extends a short distance laterally outside the distal axial shadow of introducer 101 (e.g, a distance less than one diameter of introducer 101 beyond its distal axial shadow).

For manipulator 201B, outer layer 204 is advanced longitudinally even beyond the distal-most tip of manipulator 201A. Middle layer 203 is advanced far enough that it curves to point back partially in a proximal direction, e.g., at an angle of 120°. Inner layer 202 is advance to about the same length as the inner layer 2020 of manipulator 201A. There is also a slight rotation of outer layer 204 and/or middle layer 203 of manipulator 201B so that inner layer 202 is oriented more nearly toward the distal tip of the other manipulator. Together, they define a shared action zone centered approximately on location 210 (in this case, the approximate intersection of distal longitudinal axes 211 and 212).

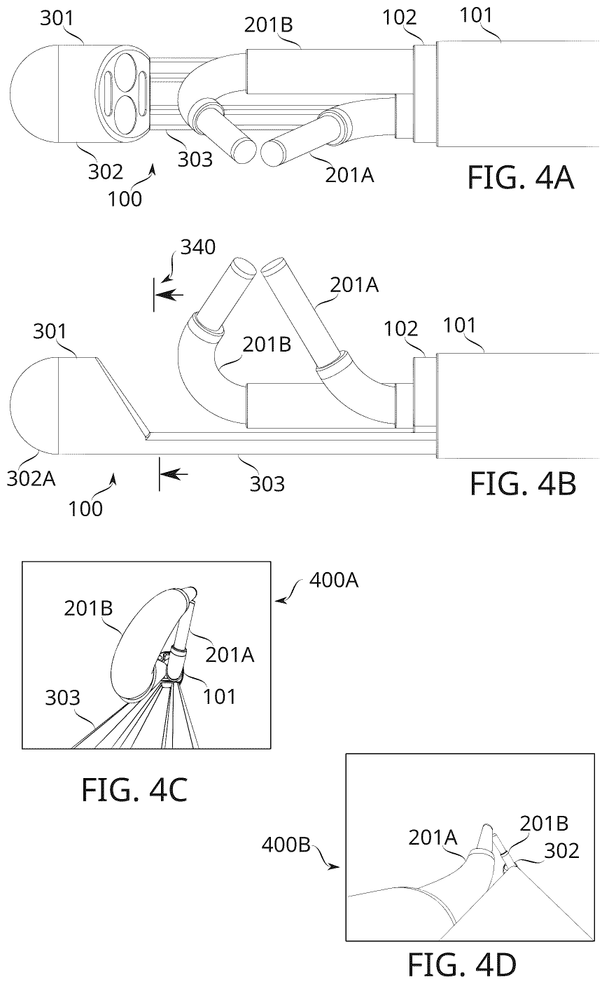

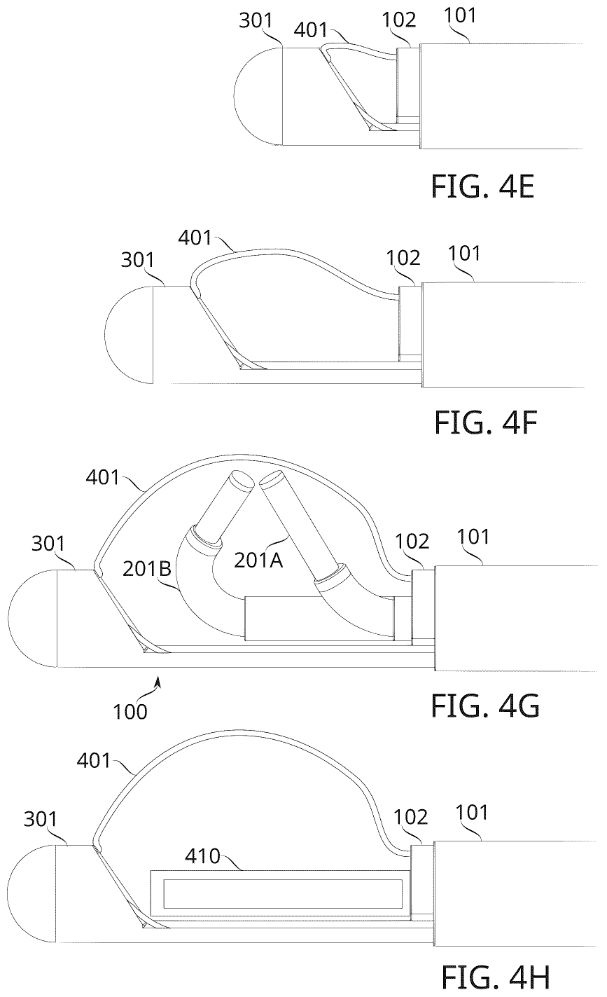

Reference is now made to FIGS. 3A-3C, which schematically represent perspective, end-on and side views (respectively) of an introducer 101 and endoscope 102 of a manipulator system 100 including a distal camera module 301, according to some embodiments of the present disclosure. Reference is also made to FIG. 3D, which shows a distal-looking view of camera pod 302 of distal camera module 301, according to some embodiments of the present disclosure. These four figures are part of a sequence of figures continuing with FIGS. 4A-4C, showing stages in the deployment of manipulator arms with system 300. The longitudinal position of the cross-section of FIG. 3B is slightly proximal to the distal terminus of introducer 101. The longitudinal position of the cross-section of FIG. 3D is slightly proximal to camera pod 302, and primarily shows features of face 308.

In some embodiments of the present disclosure, a backwards-pointing (proximal-pointing) camera angle is provided using an auxiliary distal camera module 301. Distal camera module 301 comprises camera pod 302, and pod umbilical 303.

In the example shown, pod umbilical 303 occupies most of the width of lumen 106, but is also formed with an optionally recess that allows lumen 106 to be used for advancing another tool through, for irrigation, for suction, or for another use. Channels 304 (FIG. 3B) may be used for routing electrical and/or data connections to camera pod 302. Pod umbilical 303 is not necessarily as large as shown.

Camera pod 302, in some embodiments, houses one or more cameras 305 (e.g., CMOS and/or CCD sensors with suitable lenses), and one or more light sources 304 (e.g., LED light sources). As shown, camera pod 302 is sized to fill the lumen of introducer 101, but it need not be so large as shown (e.g., approximately 9 mm in diameter, assuming a 9 mm lumen of introducer 101). It can be advanced first through the introducer, with endoscope 102 following up behind. With such a camera arrangement, endoscope 102 itself may be optional. It may be replaced, for example, with a similarly shaped device lacking cameras, optionally adjusted to take advantage of the freed-up cross-sectional area.

Optionally, camera pod 302 comprises an atraumatic tip 302A.

Reference is now made to FIGS. 4A-4B, which demonstrate a 60°/120° bend configuration of more proximally positioned manipulator 201A and more distally positioned manipulator 201B in relation to camera pod 302, according to some embodiments of the present disclosure. Reference is also made to FIGS. 4C-4D which shows distal-looking and proximal-looking views from a camera of camera pod 302 (FIG. 4C) and a camera of endoscope 102 (FIG. 4D), according to some embodiments of the present disclosure. Views 400A, 440B of FIGS. 4C and 4D are framed as images with about a 4:3 aspect ratio. Where there are two cameras distal and/or proximal to the manipulator arms 201A, 201B, views provided may be stereoscopic, or separate. It may be noted that from either point of view, the nearer manipulator may tend to obscure a portion of the field of current operations. Having camera positions distal and proximal to this field of current operations (potentially allows an operator a choice of views to allow understanding what is happening.

Reference is now made to FIGS. 4E-4G, which schematically represents a manipulator system 100 comprising a retractor scaffold 401, according to some embodiments of the present disclosure. In the example shown, retractor scaffold 401 comprises a loop of flexible rod (e.g., nitinol wire), which expands to a more open shape as it advances distally beyond introducer 101 and/or endoscope 102. Optionally, it is attached to and/or is pressed against camera module 301, so that it expands in concert with the distal advance of camera module 301. In some embodiments, retractor scaffold 401 presses against a lumenal tissue wall so as to establish a working area, and/or acts as a spacer to maintain a targeted minimal distance between arms 201B, 201A and the lumenal wall. Brief reference is made to FIG. 4H, which schematically represents a manipulator system 100 comprising an ultrasound scanning probe 410, according to some embodiments of the present disclosure. In some embodiments, ultrasound scanning probe 410 can be inserted in place of one or both of manipulator arms 201A, 201B. Optionally, rotating scanning probe 410 about its longitudinal axis redirects the ultrasound scanning plane of probe 410.

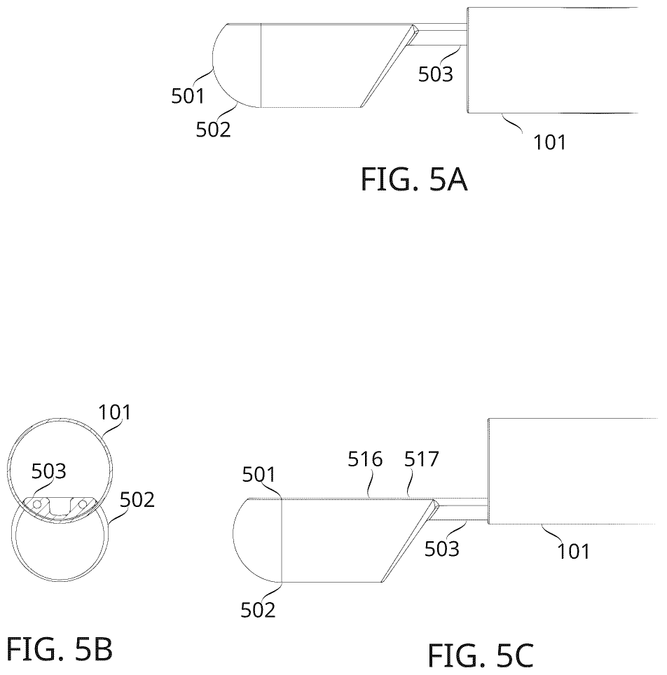

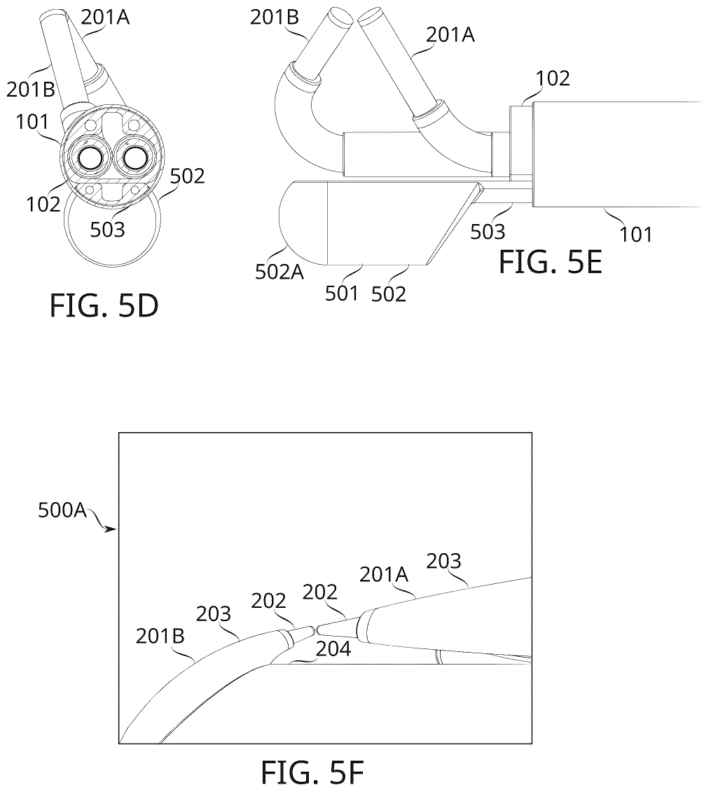

Reference is now made to FIGS. 5A-5E, which show views of a manipulator system 100 comprising a camera module 501 configured to view manipulator arms 201A, 201B from a lateral position, according to some embodiments of the present disclosure. Reference is also made to FIG. 5F, which shows a view from a camera 516, of camera pod 502, according to some embodiments of the present disclosure. View 500A for FIG. 5F is framed as an image with about a 4:3 aspect ratio.

In the configuration of FIG. 5A, camera module 501 is being advanced from introducer 101 by the distal advance of its umbilical 503. Umbilical 503 may be which may generally be shaped and otherwise configured as described for umbilical 303. However, upon initial advance, it may be positioned against the opposite lumenal wall portion of introducer 101 (e.g., with its flatter side pressed nearer to the lumenal wall). Once camera pod 502 clears introducer 101, however, umbilical 503 can move (e.g., be pressed) against the opposite luminal wall of 101. This offset places camera(s) 516 and illuminator(s) 517 of camera pod 502 near to the outer circumference of the distal axial shadow of introducer 101, with most of the remaining bulk of camera pod 502 positioned outside the distal axial shadow of introducer 101. It should be noted that camera pod 502 need not be sized to occupy the entire lumenal space of introducer 101 as shown; this may serve to limit the lateral extent of the deployed camera module 501, as may be more suitable for working in some constrained conditions.

The offset allows some distance to be created between the camera lenses and the operational region of manipulator arms 201A, 201B (the operational region may be a shared action zone), assisting in obtaining a clear view. FIGS. 5D and 5E show the system with manipulator arms extended and configured in a 60°/120° configuration, slightly rotated to once side. FIG. 5F shows a view from a camera 516 of camera pod 502. Because of their proximity to the camera, outer layer 204 and middle layer 203 of the manipulator arms 201A, 201B loom large, distorting them. They may also be difficult to obtain focus for. However, inner manipulator layers 202, being relatively distant, can be seen clearly and with relatively little distortion. This allows tools exiting their distal termini (which are also the distal termini of the manipulator arm) to be monitored in the approximate center of the viewing area.

Camera pod 502 optionally comprises an atraumatic tip 502A.

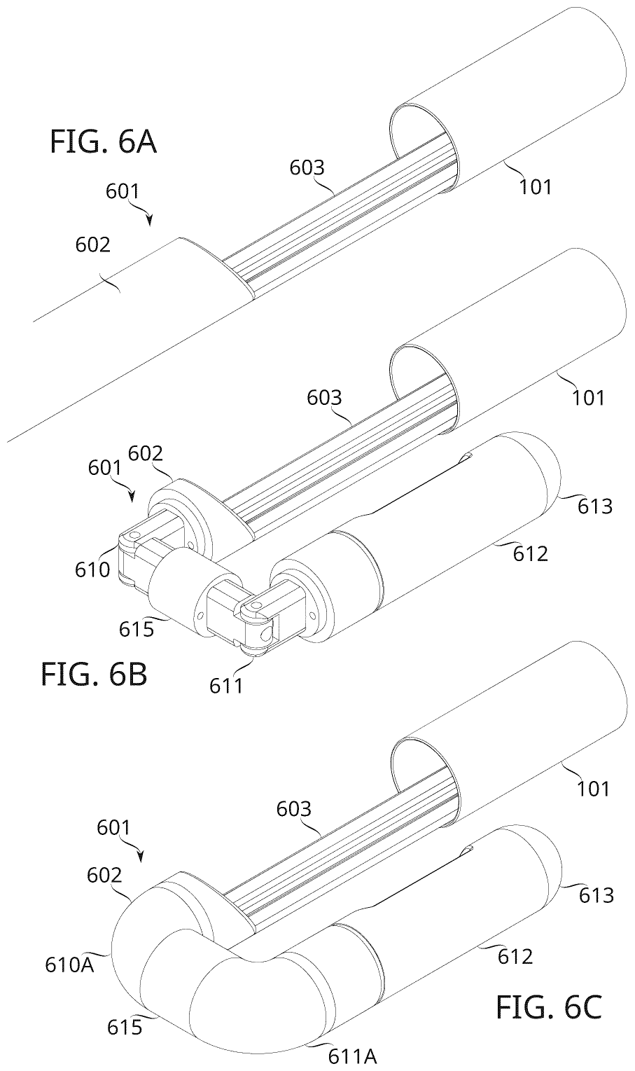

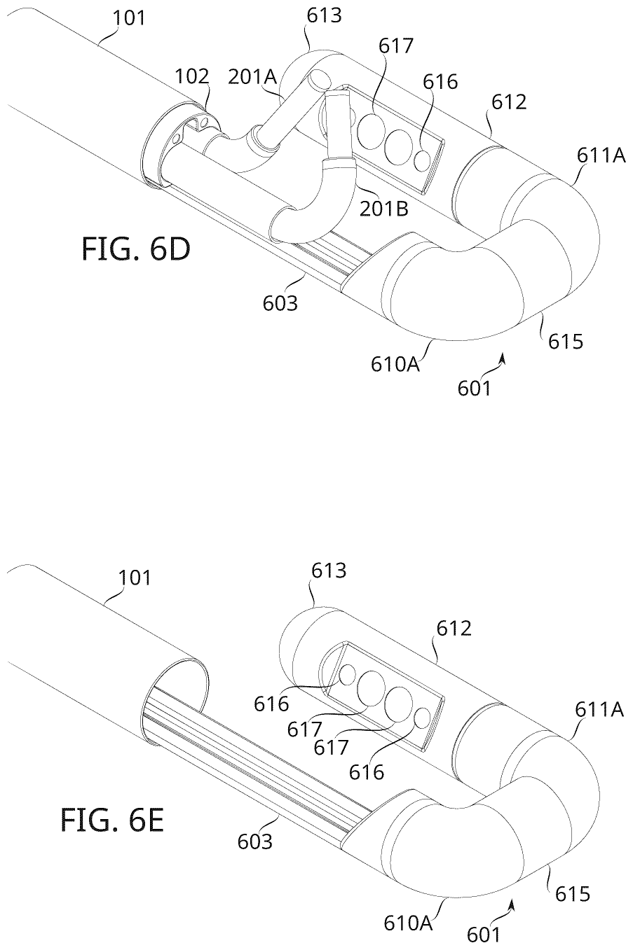

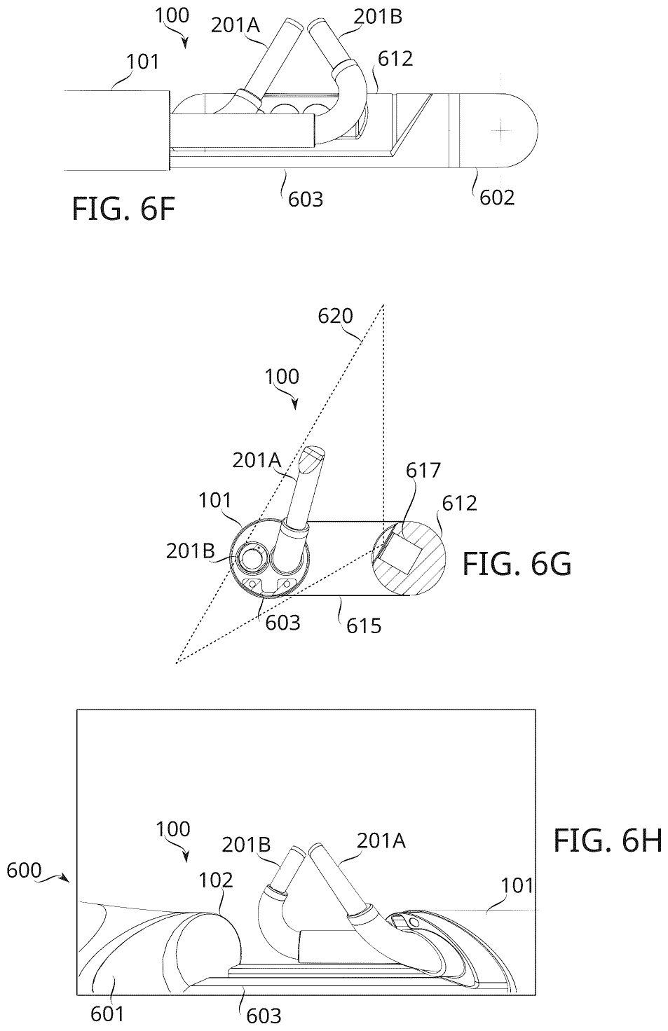

Reference is now made to FIGS. 6A-6G, which show views of a manipulator system 100 comprising a distally-mounted camera module 601, configured to bend around proximally to provide a lateral view of manipulator arms 102A, 102B, according to some embodiments of the present disclosure. Reference is also made to FIG. 6H, which shows a view from a camera 617 of camera module 601, according to some embodiments of the present disclosure.

FIG. 6A shows camera module 601 fully extended from introducer 101, and spaced from it by umbilical 603. Camera pod 602 is configured to bend around proximally, for example, to bend on joints 610, 611 via a mid-segment 615 (FIG. 6B). Optionally bending of joints 610, 611 is bidirectional (that is, the wrap-around is to the opposite side of umbilical 603). In FIG. 6B, fairings 610A, 611A (FIG. 6C) have been suppressed. Camera pod segment 612 carries one or more cameras, and optionally one or more lighting modules. It is optionally provided with an atraumatic tip 613.

FIGS. 6D and 6E show different perspective views of camera module 601, deployed and wrapped back proximally. This provides views of cameras 617 and lighting modules 616 on camera pod segment 612. Optionally, camera pod segment 612 can be rotated around its longitudinal axis, allowing the field of view to be selected with a central view axis relatively more or less lateral from the distal axial shadow of introducer 101 (e.g., elevated so that it points at the shared action zone of manipulator arms 201A, 201B; the elevation is, for example, between) 30°-45°. Optionally, the range of rotation is large enough to allow pointing at the shared action zone when camera pod segment 612 extends on either the left or right of umbilical 603. In FIG. 6E, there are no manipulator arms extended. In FIG. 6D, manipulator arms 201A, 201B are extended and positioned in 60°-forward, 120°-backward configuration. FIG. 6F shows the same configuration from a full side angle, looking across the manipulator arms toward the camera module segment 612 positioned alongside them.

In FIG. 6G, a 150° field of view angle (the view angle of FIG. 6H) is shown together with a cross-section of the system 100. The cross-section is from about mid-way along the exposed section of umbilical 603, and about mid-way through camera module segment 612. Image 600 of FIG. 6H shows the resulting view (framed as an image with about a 16:10 aspect ratio), which again places the operational region (in this case also a shared action zone) of manipulator arms 201A 201B near the center of the field of view. Optionally, a camera with 30 another view angle is used, for example, with a view angle of 120°, 105°, 90°, or another view angle.

### Surgical Manipulator Arm System Positioning Examples

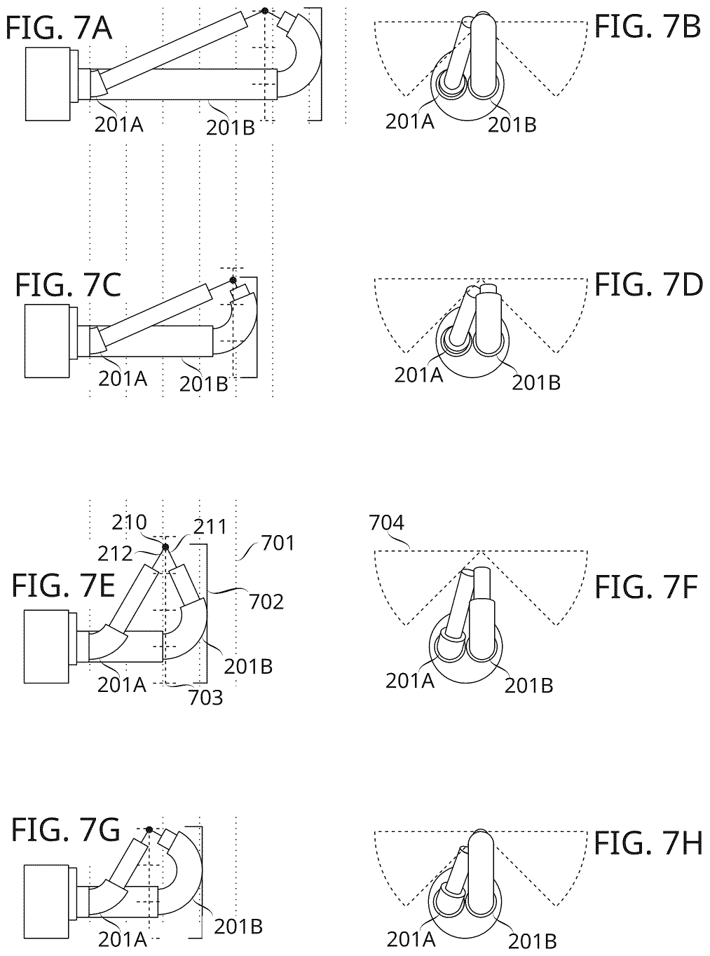

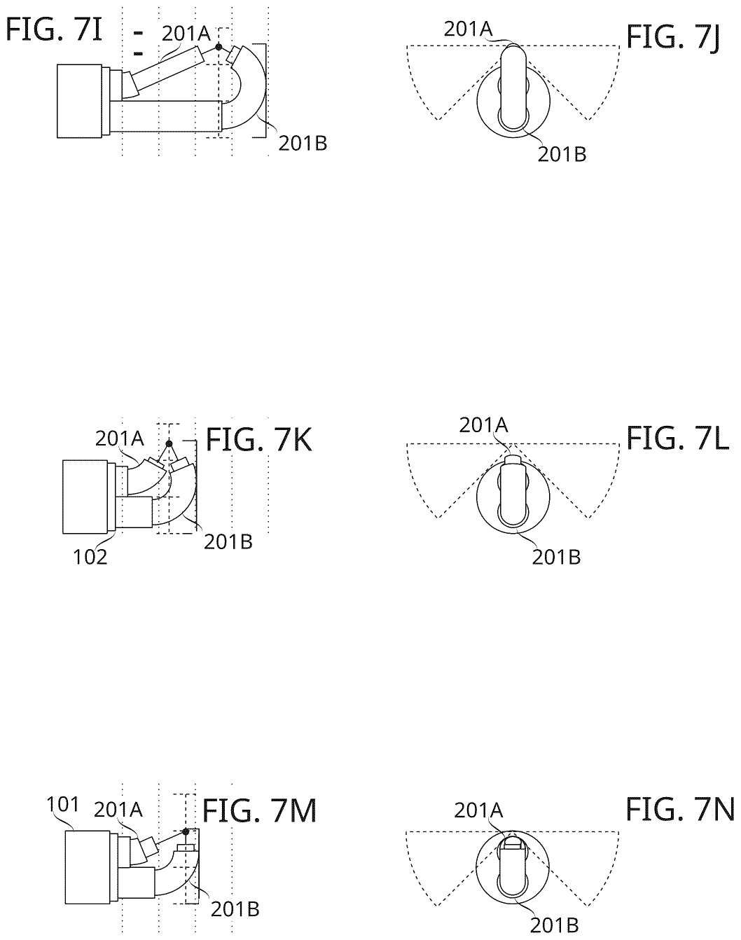

Reference is now made to FIGS. 7A-7N, which illustrate a variety of positioning configurations of manipulator arms 201A, 201B, according to some embodiments of the present disclosure. For clarity, the drawings are simplified, but they may be considered, for example, to be examples of positions which may be achieved with, e.g., any of the system configurations of Figures IA-6H.

Arm positions shown in Figures such as FIG. 2A, FIGS. 4A-4D, FIGS. 5D-5F, and FIGS. 6D and 6F-6H are in an approximate “equilateral triangle” position, with the proximal arm articulated at about 60°, and the distal arm articulated at about 120°. In some cases, they are slightly rotated about the distal longitudinal axis of the introducer 101, which may help bring tools exiting the arms together, and/or may assist in getting a more side-on view from certain camera positions. This configuration roughly corresponds to what is shown in FIG. 7E (looking from the side) and FIG. 7F (looking proximally).

To assist in comparison of the various configurations, vertical dotted lines 701 are superimposed on FIGS. 7A, 7C, 7E, 7G, 7I, 7K, and 7M. The dotted lines are each separated by about one radius length of introducer 101 (e.g., about 5 mm). These lines are referenced to the longitudinal position from which positioning steering of the manipulators can begin. This position is optionally withdrawn proximally at least to the distal lip of introducer 101, but in the figures is shown somewhat distally advanced for purposes of illustration.

A radial scale 703 is also provided; again, each mark on the scale is separated by about one radius length of introducer 101. Dots 210 in these figures show the estimated position of the center of a shared action zone created by two manipulator arms at their depicted positions. Distal longitudinal axes of each manipulator arm 201A, 201B are also shown as axes 212, 211, and meet at the shared action zone center. Overall radial extent of the system 100, extending to the center of the shared action zone, is indicated by a bracket 702 on each of these figures. 25 Numbering of some elements is suppressed in most figures to reduce clutter, but examples may be seen, for example, in FIGS. 7E, 7K, and/or 7M.

The end-on (proximal-looking) views of FIGS. 7B, 7D, 7F, 7H, 7J, 7L, and 7N each correspond to the configuration in the drawing to their immediate left. The circle segments 704 (labeled in FIG. 7F) suggest a range of lateral angular positions at which a camera may be positioned in order to provide a relatively unobstructed view on to the corresponding shared action zone center.

In each of FIGS. 7A-7H, the manipulator arms 201A-201B bend into roughly parallel planes. This mode of operation may require somewhat more lateral and/or longitudinal room to reach a targeted shared action zone center than in the examples of FIGS. 7I-7N, wherein both manipulator arms substantially share the plane within which they bend. The reason for this is that the more distal manipulator arm 201B may require somewhat more room to curve-since it curves through a larger angle. Locating manipulator arm 201B at the side opposite its direction of curvature gives it more “in shadow” room. The penalty for this is that the more proximal manipulator arm 201A has less room to curve. However, this may still result in a shorter lateral displacement distance on balance.

In the examples of FIGS. 7A-7B and in the example of 7E-7F, the bends of each manipulator arm 201A, 201B are each about the same number of degrees different than 90° (e.g., 30° on the proximal side, and 150° on the distal side). This gives both manipulator arms approximately equal leverage when worked against each other. The relative high obliquity from) 90° (60° also helps to keep the center of the shared action zone relatively near to the distal axial shadow of introducer 101; in this case, within about a radius of introducer 101. However, this requires greater longitudinal space. In this example, the center of the shared action zone is about 5 radius lengths away from the location from which steering is controlled. The maximum longitudinal extent needed by the system is a bit longer than this, since the more distal manipulator arm 201B needs space to extend distally past and then proximally back toward the center of the shared action zone.

In the example of FIG. 7C-D, manipulator arm 201B is bent less; by about 120°. While this makes the two manipulator arms 201A, 201B operate at different angles, it has the potential advantage of moving the center of their shared action zone both slightly more inward, and slightly more proximal.

The configuration of FIGS. 7E-7F has the largest lateral shared action zone displacement of the examples shown. With angles less oblique to 90°, it could be displaced still further laterally. In this example using a particular radius of curvature and particular manipulator arm thickness, the lateral displacement is about 1.3 diameters from the central longitudinal axis of the introducer 101, at a minimum longitudinal displacement of just about one diameter.

In FIGS. 7G-7H, manipulator arm 201B is again bent by about 150°, but now manipulator arm 201A is bent by about 60°. This allows pulling the center of the shared action zone further proximally than in the situation of FIG. 7A, at the expense of changing the relative leverage angles of the two manipulators.

In all of FIGS. 7A-7H, a lateral camera viewing angle may advantageously be placed, for example, as shown for the embodiment of FIGS. 6A-6H, in order to obtain a view onto the device similar to that shown in FIGS. 7A, 7C, 7E, and 7G. The angular zones 704 of FIGS. 7B, 7D, 7F, and 7H illustrate that the range of lateral camera positions available with up to about 45° obliquity includes positions which may be easily reached by configurations such as the embodiment of FIGS. 6A-6H.

Additionally or alternatively, a “below the plane” view such as is provided by the embodiment of FIG. 7A-7F may be used, noting that there is a potential need to maintaining a clear focus for each of these different configurations. This may be met, for example, by providing focus adjustment in the camera pod. Also, the amount of distortion may be quite different depending on how far away or close the working area is. The region of camera placement is not illustrated for this option. It may be preferable to place the camera somewhat on the side of the more proximally positioned manipulator arm 201A, to reduce blocking interference from the pre-bend region of manipulator arm 201B.

The examples of FIGS. 7I-7N may be understood as showing the system of manipulator arms rotated along its longitudinal axis by 90°, compared to the configuration of FIGS. 7A-7H. Put another way, if FIGS. 7A-7H show views from the right side of the system, FIGS. 7I-7M show views from its bottom. This results in the angular zones 704 of FIGS. 7J, 7L, and 7N being indicative of where positions of cameras “below” or “above” the system would obtain clear views (and more lateral camera positions are not shown). Again, camera locations are not limited to angles within the ranges shown; the angle ranges shown are only indicative.

Comparing FIG. 7I to FIG. 7A, and FIG. 7K to FIG. 7E, it may be seen that suitable axial rotation of the manipulator arms potentially reduces the amount of working space required in order for the two arms to define a shared action zone center. The configuration of FIGS. 7M-7N even generates a center of a shared action zone which is about at or even within the distal axial shadow of introducer 101, and yet has a difference in approach angle between the two manipulator arms of about 60°. However, one of the manipulators here works completely in a lateral direction. To a certain extent, the center could be brought even further radially inward by bending manipulator 201B less than 90°, eliminating proximal-pointing. However, this extremely close-in work is potentially limited in utility, since there is correspondingly little room to retract. For example, if a tool extended from manipulator arm 201B grips tissue and then retracts to expose tissue to a cutting operation from a tool in manipulator arm 201A, it may not be have room to retract enough distance to expose the tissue as desired. Stitching actions may also be awkward. On the other hand, this close-in configuration may be well suited to other operations; for example, wherein one manipulator arm provides suction to clear fluids, and the other holds a tool used to generate lesions by non-mechanical manipulation (e.g., using laser or RF energy). These examples serve to illustrate more generally that the minimum required working space for an action is not necessarily defined only by how much space is need to reach a target; there should also, in some cases, be sufficient space to perform actions such as stretching, separating, pulling, and tightening. In those types of cases, it may be useful to be able to select between, pulling mostly along a longitudinal axis (e.g., as in FIG. 7A), or pulling more nearly along a lateral axis (e.g., as in FIG. 7E). The ability to choose either (e.g., as in FIG. 7G), may also be of potential benefit.

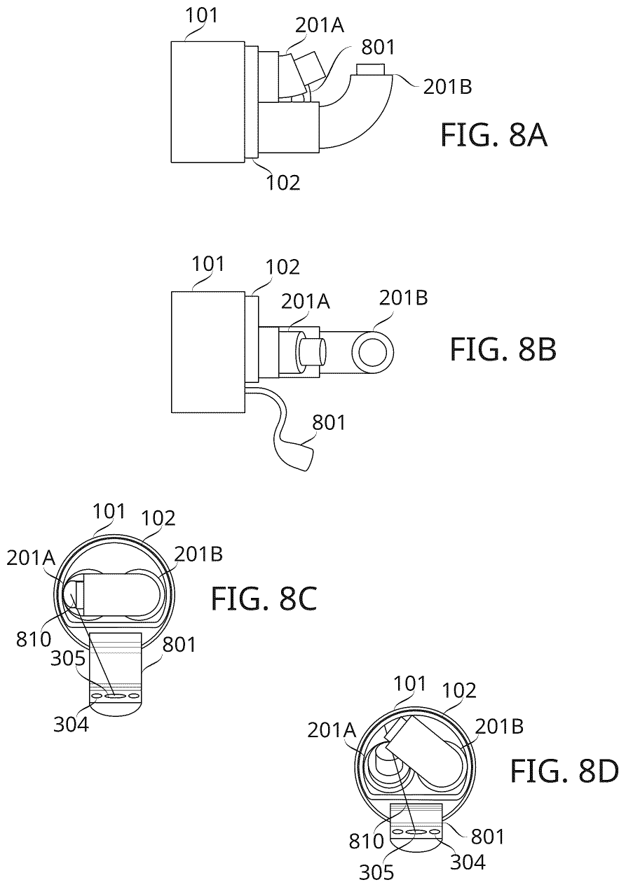

Reference is now made to FIGS. 8A-8D, which schematically illustrate how relationships between camera position and arm position may be adjusted to maintain a suitable working distance between camera and operational region. FIG. 8A shows the manipulator arms 201A, 201B in the configuration of FIG. 7M, with the addition of a camera module 801 which is located (as shown in FIG. 8A) so that it looks onto the bending plane of the two manipulator arms from position laterally offset out of the distal axial shadow of introducer 101 by an umbilical. The umbilical may be flexible to allow it to be fully withdrawn into introducer 101, or it may simply be shaped as shown, and advanced beyond introducer 101 before advancing other components including manipulators arms 201A, 201B into position. The widened (e.g., bulbous) extension of camera module 801 houses the camera and/or lighting. It may be larger than shown; the point is the position of camera(s) 305 and/or illuminator 304, relative to the distal ends of manipulator arms 301A, 201B.

FIG. 8C may be compared with FIG. 8D. In both cases, camera 305 is about the same distance (distance 810) from the center of the shared action zone which the two manipulator arms 201A, 201B define. However, in FIG. 8D, rotating the two arms to a slightly different position allows the camera 305 to be pulled significantly radially inward (“upward”, in the end-on orientation shown). The rotation of the manipulator arms 201A, 201B is purely around the axes of their introductory trajectories.

### Use of Surgical Manipulator Arm Systems

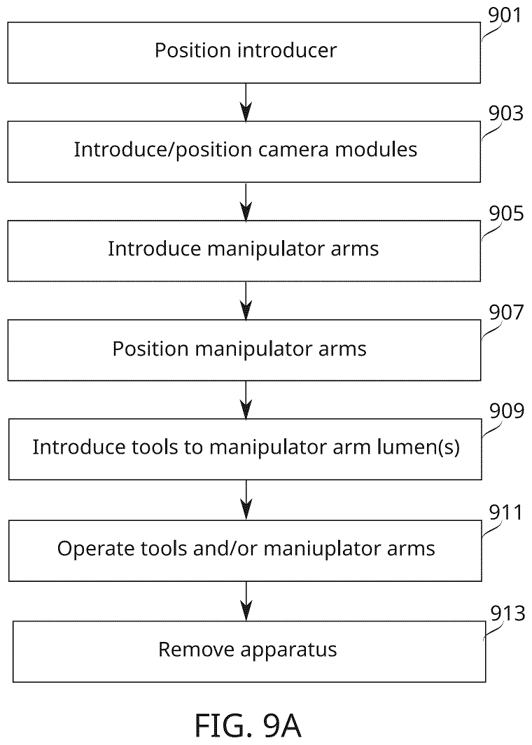

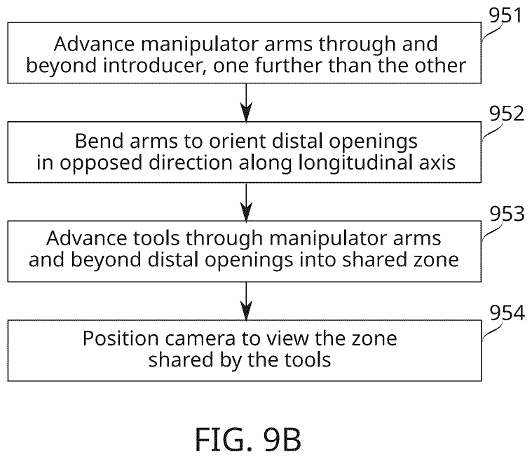

Reference is now made to FIG. 9A, which is a schematic flowchart of a method of positioning manipulator arms, according to some embodiments of the present disclosure. The manipulator arms may be, for example, manipulator arms 201A, 201B.

At block 901, in some embodiments, the flowchart begins, and an introducer (e.g., introducer 101) positioned where it will give the manipulator arms access to a surgical field.

Optionally, at block 903 one or more camera modules are advanced through the introducer, and positioned as appropriate to their design. The camera modules are configured to be positioned where they will provide suitable views for monitoring operations of the manipulator arms and/or tools passed through them. For example, they may be positioned distal to the arms, or lateral to them (e.g., radially away from the introducer's distal axial shadow, and looking toward and/or across it). Lateral positioning may be to any radial direction (e.g., left/right/top/bottom, if such directions are defined). A camera module is provided with an umbilical sufficiently small that it will not prevent later advancing the manipulator arms into 1.5 position. Positioning of some camera module designs is optionally adjusted at any later stage of the procedure, so as to visualize operations of manipulator arms and/or tools, as appropriate.

At block 905, in some embodiments, the manipulator arms themselves are introduced into the surgical field. They are optionally introduced over a lumen of an endoscope which also passes in through the introducer. However, the lumen need not be that of an endoscope (e.g., it can be an auxiliary introducer). There does not need to be even an auxiliary introducer in some embodiments, but use of some kind of secondary lumen has the potential advantage of maintaining a known and/or preferred alignment of components such as camera, camera umbilical, and manipulator arms. In some embodiments, the manipulator arms share a lumen of the endoscope/auxiliary introducer, and are sized relative to that lumen such that they are constrained to run in contact with each other along parallel axes.

At block 907, in some embodiments, the manipulator arms are positioned. In some embodiments, positioning comprises asymmetrical positioning of the manipulator arms. The asymmetrical positioning may comprise:
advancing a first manipulator arm into the surgical field, and then articulating it by a bend of less than 90°.
advancing a second manipulator arm distally past the first manipulator arm's distal end, and then bending it back more than 90° through a bend in the second manipulator arm.

During positioning, the first and second manipulator arms are introduced, in some embodiments, along parallel axes (their introductory trajectories). Their bending, for distal portions having longitudinal axes leaving this introductory trajectory, may be solely in one direction. The bending may be continuous through a single bendable region. The bending may leave the manipulator arm in a co-planar configuration; for example, if there is only one bend, all parts of the manipulator arm remain in the same plane after bending. The bending is optionally through a constant radius of curvature, for example, a radius of internal curvature which is at least half the radius of the region that bends, and optionally not more than twice the radius of the region that bends.

In some embodiments, the manipulator arms comprise three layers, of which the more outer and more inner layers are stiff and extend straight when unconfined. The middle layer can assume a straight configuration, but it may be predisposed to assume a bend when unconfined and (e.g., elastically predisposed to assume a bend when unconfined, and/or under tension from a member which pulls on it to urge it into a bent configuration). Optionally, the middle layer is bendable to a varying degree, depending on how much tension is placed on it from a pulling member. The inner layer is sufficiently flexible to pass through the middle layer, but is predisposed to straighten again where it has passed distally out of the middle layer. Straightening/bending forces operating on the middle and inner layers are balanced so that the inner layer does not overcome the bending forces of the middle layer by its own tendency to straighten.

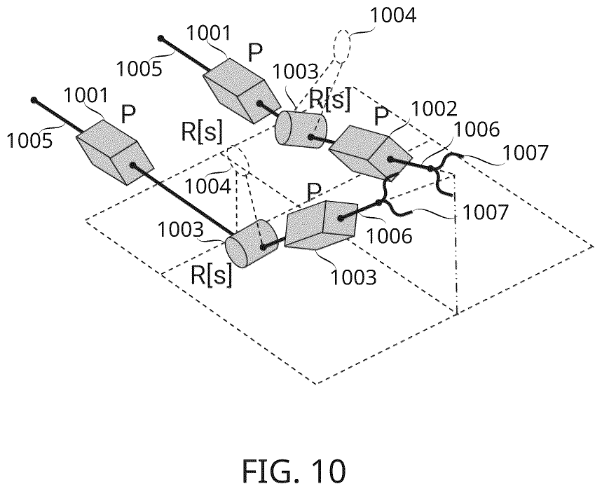

It should be understood that embodiments herein are not limited to the three layer construction just described, or as described elsewhere herein. FIG. 10 provides a more general characterization of manipulator arms used in some embodiments of the present disclosure, described in terms of degrees of freedom of movement.

Optionally, positioning of the manipulator arms is performed under observation by one or more cameras, e.g., one of those introduced in block 903, and/or one or more cameras of an endoscope used to assist introduction of the manipulator arms.

The manipulator arms are optionally positioned so that they terminate distally where the ratio of their radial (lateral) distance from a central distal longitudinal axis of the introducer, and their longitudinal distance from the distal end of the introducer is at least 0.3, at least 0.4, at least 0.5, at least 1, or at least 1.5. Optionally, the manipulator arms are positioned with a distal end positioned no more than 5 radius lengths of the introducer along a distal longitudinal axis of the introducer (that is, measured from the distal end of the introducer). Optionally, the manipulator arms are positioned with any of the relative distances and/or ratios shown in FIGS. 7A-7N, with the diameter of the introducer being 10 mm or another diameter, for example, 7 mm, 9 mm, 12 mm, 15 mm, or 20 mm.

At block 909, in some embodiments, respective tools are introduced into one or both of the manipulator arms, up to and optionally beyond their distal ends. The manipulator arms are provided with interior lumens, sized to allow passage of their respective tools.

Optionally, at block 911, in some embodiments, one or more of the cameras introduced in block 903 are used to monitor operations of the tools from a position lateral to the working region of the tools. The lateral position may also be longitudinally alongside the working region of the tools; e.g., within an arc of about +30° of an axis running perpendicular to a distal longitudinal axis of the introducer. Tool operation is optionally conjoined with movements of the manipulator arms themselves. Here and in block 907, operation of the manipulator arms may comprise direct operation of them by a human operator via mechanical linkage. Additionally or alternatively, operation may be motorized. Motorized operation may be under the control of commands generated for a motor controller based on human operator inputs, and/or under the control of commands generated automatically, for example, according to a surgical plan and/or selected target.

At block 913, in some embodiments, any of the tools, manipulator arms, camera modules, auxiliary introducers, endoscopes, and introducers positioned in the vicinity of the surgical field in the course of blocks 901-911 is removed. The flowchart ends.

Reference is now made to FIG. 9B, which is a schematic flowchart of a method of positioning manipulator arms, according to some embodiments of the present disclosure. The manipulator arms may be, for example, manipulator arms 201A, 201B. In some embodiments, operations of FIG. 9B are included as elements of the operations of FIG. 9A, for example, related to the positioning of manipulator arms, camera(s), and advancing of tools to a site through lumens of the manipulator arms.

At block 951, in some embodiments, first and second manipulator arms are advanced through and distally beyond an introducer, and along a distal longitudinal axis of the introducer. The manipulator arms are positioned so that the second manipulator arm is advanced distally further than the first manipulator arm.

At block 952, in some embodiments, the first and second manipulator arms are bent to orient respective distal openings of each manipulator arm to opposing directions along the distal longitudinal axis. The distal openings may be adjusted so that they open toward each other. In some embodiments, the arms are bent within a plane.

At block 953, in some embodiments, a respective tool is advanced out of the respective distal opening of each of the manipulator arms into a shared zone of action (shared action zone) defined by the relative positioning of the two manipulator arms.

At block 954, in some embodiments, a camera is introduced by advance distally from the introducer, and positioned so that it has with a field of view including the shared zone of action.

### Surgical Manipulator Arm SystemDegrees of Freedom

Reference is now made to FIG. 10, which schematically illustrates degrees of freedom of the joints of a pair of manipulator arms, according to some embodiments of the present disclosure. In some embodiments, each manipulator comprises a first prismatic joint 1001 (allowing extension and retraction), a second prismatic joint 1002 (also allowing extension and retraction), and revolute joint 1003, optionally having an offset center 1004. Optionally, additional degrees of freedom (not shown) allow rotation around the longitudinal axes of elements 1005 and/or 1006, respectively, in which case the prismatic joints 1001, 1002 may be replaced with cylindrical joints.

Each tool 1007 may provide one or more additional degrees of freedom; for example, a pincer movement, or another motion.

### Other Surgical Manipulator Arm System Features/Concepts

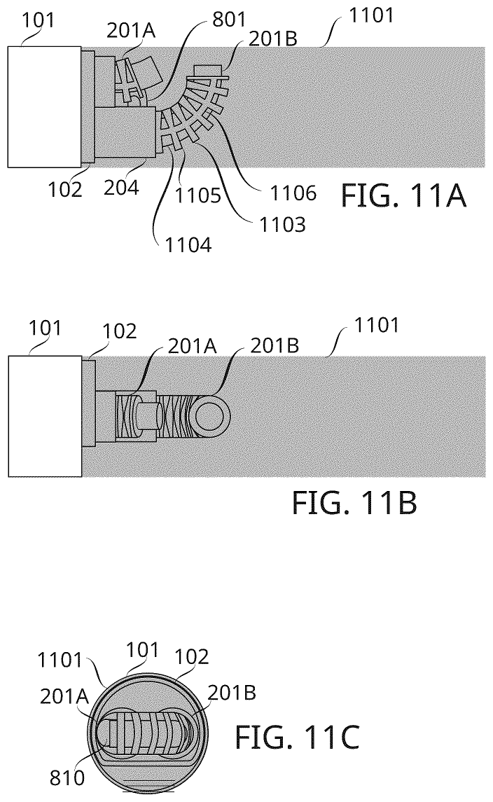

Reference is now made to FIGS. 11A-11C, which schematically illustrate the extent of a distal axial shadow 1101 of an introducer 101, according to some embodiments of the present disclosure. The shaded area in each of the three figures indicates the extents of the distal axial shadow as seen from three different viewpoints. In the configuration shown, both manipulator arms 201A, 201B remain fully within distal axial shadow 1101. Optionally, they may be advanced radially beyond it. Formally, there is no necessarily defined distal end to the shadow (it continues indefinitely), but the distal side of the introducer 101 does mark the proximal terminus of the distal axial shadow.

Also indicated in FIGS. 11A-11C is an optional construction of a middle layer 1103, which may be used to provide the middle layer 203 of any of the embodiments illustrated in other figures herein. Middle layer 1103 is slit by a plurality of slits 1104, with the inter-slit regions 1105 being joined by thin strips of joining material 1106. There may be, for example, two strips 1106 joining each sequential pair of inter-slit regions 1106, the strips 1106 being separated from each other by a slit 1104 on both the inner side of the curvature of middle layer 1103, and on the outer side of the curvature of middle layer 1103. Alternatively, there may be one joining strip, e.g., on the outer curvature of middle layer 1103. Bending of middle layer 1103 as it advances from outer layer 204 may be due to self-bending, e.g., middle layer 1103 may be of nitinol construction, preconfigured to bend into a curved shape when unconstrained to another shape. In some embodiments, the curvature is limited by self-interference, e.g., contacts among inter-slit regions 1105 on the inner curvature of middle layer 1103 set the maximum amount of bending. Additionally or alternatively, this construction may be used with a tensioning wire connected to a distal end of middle layer 1103. Upon tensioning of the tensioning wire, curvature of middle layer 1103 is induced, wherever middle layer 1103 is unconstrained, e.g., by outer layer 204.

### Robotic Systems

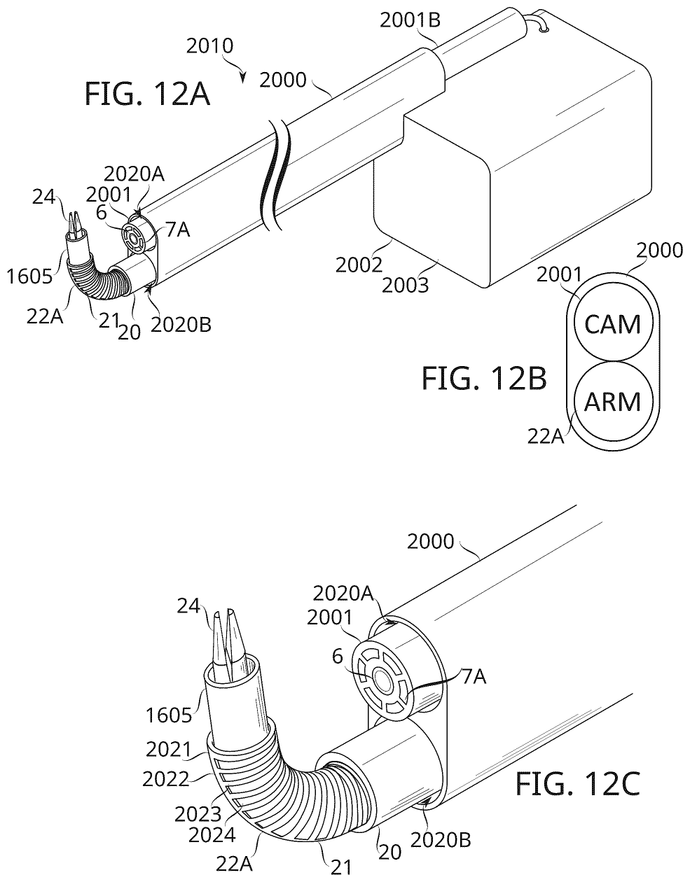

Reference is now made to FIGS. 12A-12C, which schematically represent a configuration of a modular robotic endoscope system 2010, according to some embodiments of the present disclosure. FIG. 12C is a magnified view of a distal region of elements shown in FIG. 12A. The illustrated embodiment of system 2010 is configured to provide separate body-inserted elements comprising endoscope 2001, and steerable working channel 22A. FIG. 12B presents a schematic end-on view of the arrangement of these elements, with endoscope 2001 also labeled CAM (for “camera”), and steerable channel 22A labeled ARM (for “robotic arm”). It should be understood, both generally and particularly with respect to modular aspects of embodiments shown in FIGS. 12A-15C, that embodiments of the present disclosure are not limited to camera and arm configurations shown; they are provided as examples.

Introducer 2000 is preferably straight and stiff, e.g., suitable for stable, predictable and/or reproducible positioning, for example as is required of many neurosurgical procedures. Introducer 2000 comprises two working channels 2020A and 2020B. These may be (but are not necessarily) identical in size and shape. This can promote flexibility and modularity; e.g., so that the CAM and ARM positions can be swapped and/or duplicated.

Steerable channel 22A may be a manipulator arm, for example as described in relation to manipulator arms 201A, 201B of FIGS. 3A, 4A, 7A-7N, 8A-8D, 11A-11C, and/or in relation to other figures herein. It is shown equipped with bi-polar tool 24, but any other tool may be optionally provided. In the example shown, middle channel tube 21 is implemented more particularly as a slotted tube 2021 (FIG. 12C). In this example, at least one spine 2022 of the slotted tube interconnects rings 2023, spaced apart by slots 2024. Slotted tube 2021 is optionally elastically biased (e.g., spring-annealed) to assume a curved shape when unconstrained, while being sufficiently flexible to straighten, e.g., upon withdrawal into outer channel tube 20. Optionally, an additional inner channel tube 1605 is provided, although a tool such as bi-polar tool 24 may be sufficiently self-supporting that inner channel tube 1605 is omitted.

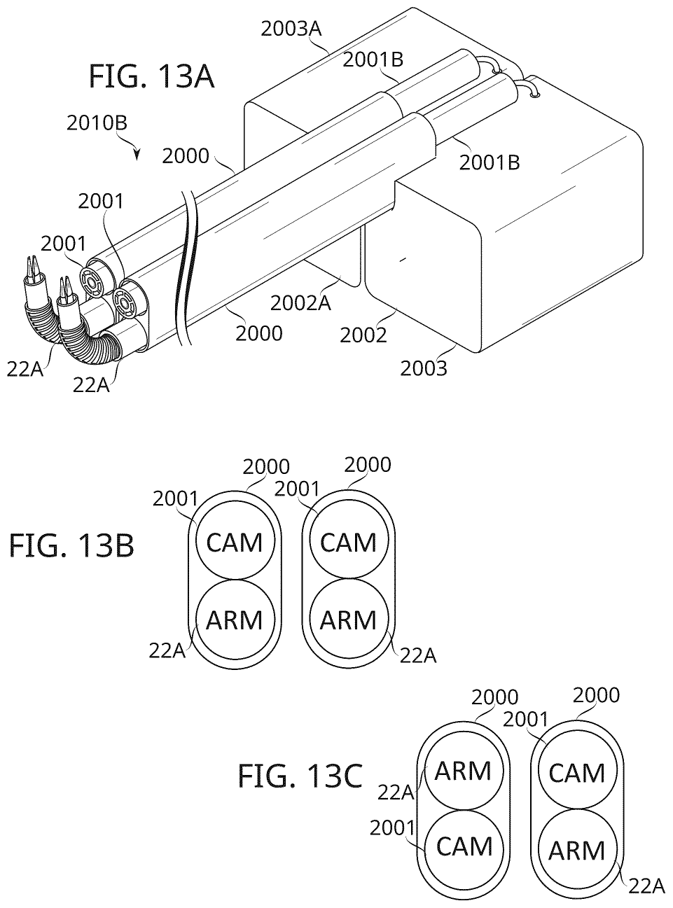

At a proximal side, steerable channel 22A interconnects with robotic controller 2002. Introducer 2000 connects with the enclosure 2003 of robotic controller 2002, which in turn is configured to operate steerable channel 22A. In some embodiments, connection of introducer 2000 and robotic controller 2002 positions proximal-side regions of elements of steerable channel 22A along a side and/or corner of enclosure 2003. Optionally, these elements of steerable channel 22A are positioned with their own cross-sectional areas at least partially, and optionally completely within the proximal-distal axis profile of enclosure 2003 (that is, a profile of enclosure 2003 as seen from a distal-side position). The side and/or corner positioning of introducer 2000 with respect to the enclosure 2003 of robotic controller 2002 potentially allows side-by-side and/or radially arranged configurations using more than one robotic controller 2002, for example as described in relation to FIGS. 13A-15C.

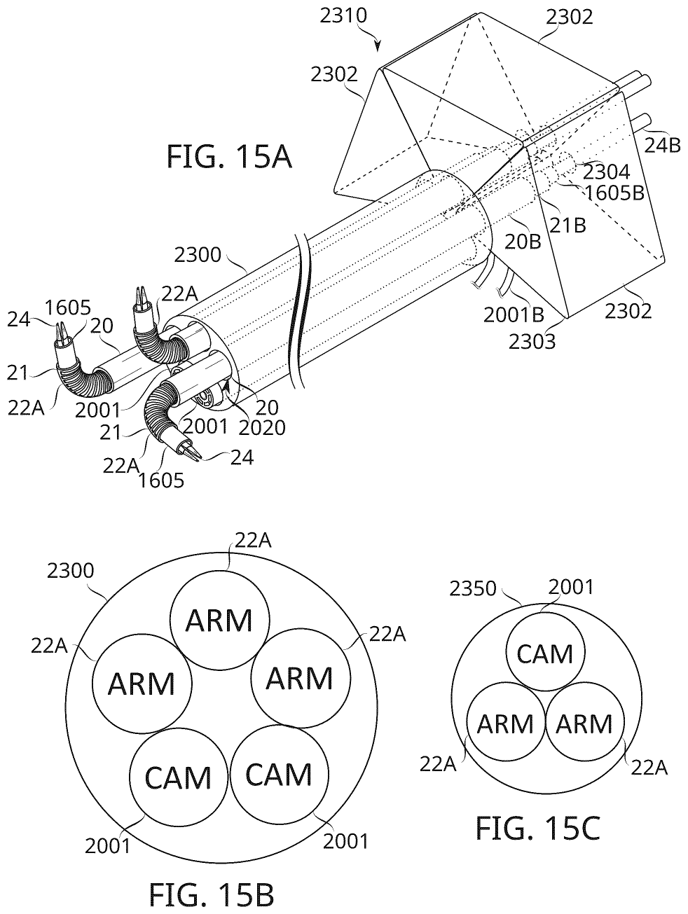

The mechanics of robotic controller 2002 are arranged to engage one or more of the elements of steerable channel 22A, and to actuate their movements (e.g., distally/proximally, and/or rotating). Optionally, robotic controller 2002 also includes actuators for tools, for example, to operate the pincers of bi-polar tool 24. In some embodiments, one or more actuatable elements of steerable channel 22A pass through robotic controller 2002, e.g., to a more proximal module, or to allow direct manual control. Optionally, tool passthrough is provided of actuating element such as cables, wires and/or rods. Apart from its use in actuation, passthrough may be used to provide access to withdraw and insert elements of channel 22A and/or tools used with it, e.g., to exchange elements and/or tools. Passthrough is illustrated, for example, in FIG. 15A.

Endoscope 2001 may comprise any suitable endoscopic capabilities; as shown, it is provided with a camera lens 6 (equipped also with a camera), and illuminator array 7A. Endoscope 2001 can be advanced or retracted through its working channel 2020A by manipulation from a proximal end 2001B. It is shown interconnected with robotic controller 2002 for receiving power/commands for the imaging devices, and/or returning data to robotic controller 2002. These connections are optional, e.g., power may be separately provided, and/or imaging results may be displayed without passing (or at least, not passing directly) into robotic controller 2002. As shown, movements of endoscope 2001 are not themselves robotically controlled, although optionally endoscope 2001 is provided with its own robotic controller, configured for operating its particular degrees of freedom.

Reference is now made to FIGS. 13A-13B, which schematically represent an expanded configuration of a modular robotic endoscope system 2010B, according to some embodiments of the present disclosure. In overview, the elements of system 2010B are the same as for system 2010, except that optionally enclosure 2003A is mirrored with respect to enclosure 2003. To accommodate this change, some portion of the mechanics of robotic controller 2002A are also mirrored. Another option is to design enclosure 2003 so that it can interface equivalently with introducer 2000 in any of at least two orientations which differ from each other by a rotation of 90°. In this case, introducer 2000 may protrude from the “top” of enclosure 2003, or from its “side”, and in the latter case, enclosure 2003 can be rotated to make that side its new top. The arrangement shown allows compact-side-by side positioning of two sets of endoscope and arm, in the same relative orientation e.g., as illustrated in FIG. 13B. The closeness of placement of the robotic arms (e.g., steerable channels 22A) is, for example, limited only by the wall thickness of introducers, while allowing them also to be parallel, e.g., so that they can share a single access way to a target site.

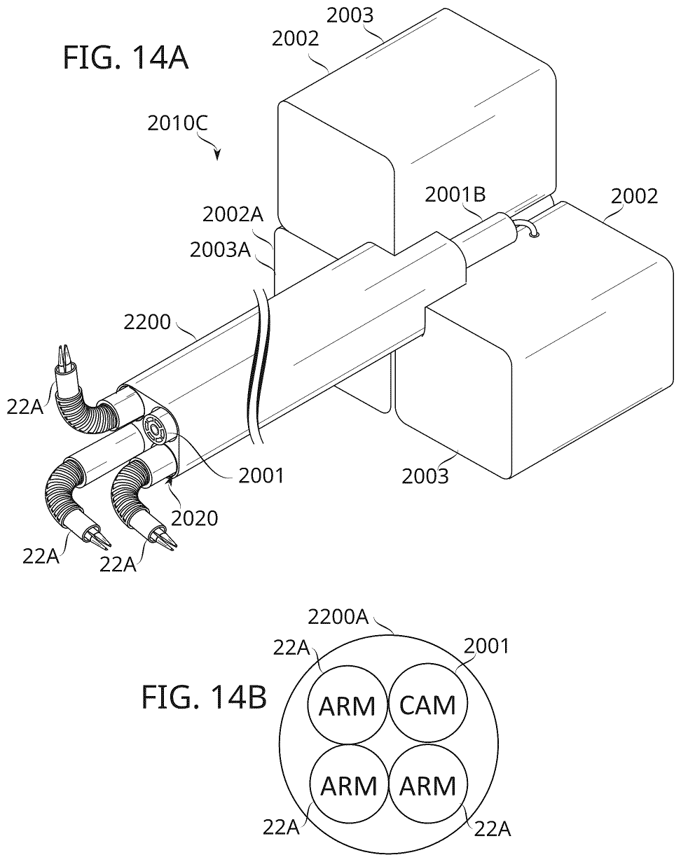

Brief reference is now made to FIG. 13C, which schematically represents an alternative expanded configuration of a modular robotic endoscope system 2010B, according to some embodiments of the present disclosure. In one of the introducers 2000, the positions of ARM and CAM is shown flipped. This allows a second copy of enclosure 2003 to be placed inverted and offset alongside the first enclosure 2003, without a requirement for mirroring its design, or for supporting mating with introducer 2000 in more than one relative orientation. This positioning may be visualized with respect to FIG. 14A, and the two enclosures 2003 positioned at opposite corners of introducer 2200.

It is an aim of some embodiments of the present disclosure to flexibly provide a capability for “manipulator density”; that is, to allow bringing a plurality of stiffly-supported robotic manipulators along parallel routes through a space-constrained passageway. A need for parallel routes may arise in part due to the use of straight and stiff introducers, e.g., for reasons as described in relation to Figures IA-1B.

The enclosure 2003 of robotic controller 2002 is optionally completely self-contained in the role of motion controller. For example, it receives commands in the form of instructions abstracted from hardware specifics, converts these into lower-level commands suitable for components such as motors, and also contains the motors and interfacing hardware (e.g., gears, cables and/or other mechanics which actually contact and move elements such as proximal-side portions of the elements of steerable channel 22A. Sensors (e.g., cameras and/or encoders configured to track and/or verify movement) are optionally provided. Being self-contained may promote modularity and/or simplicity of set-up.

When enclosures are closely arranged, e.g., side-by-side as in FIG. 13A or in another fashion such as is described in relation to FIGS. 14A-15C, there is physically plenty of room in directions radially away from their common center to put all this hardware. Use of this room need not maintain the square aspect ratio shown for enclosures 2003; e.g., the enclosures 2003 can be rectangular, or another shape (for example, FIG. 15A shows a roughly triangular enclosure shape).

However, there may be other constraints on available space for enclosures, e.g., constraints on their weight, or constraints stemming from a need to access the patient in other ways as well.

Accordingly, in some embodiments of the present disclosure, the elements of robotic controller 2002 which are provided within enclosure 2003 may be only a portion of the elements of robotic controller 2002. For example, the contents of enclosure 2003 may implement only what is mechanically needed to move elements, without control logic, and optionally even without motors. Sensor reading and/or external control logic may be implemented, e.g., by a microcontroller or other computing device; communicating as necessary with elements inside enclosure 2003 via a suitable wired or wireless data link. Motor force may be provided from an external motor through a linkage, e.g., a rotating cable. Distributing at least some functions of robotic controller 2002 to enclosures away from enclosure 2003 may assist in achieving a smaller size in locations where space constraints are the most limiting. Although potentially more complex to implement, modularity of design is also possible here, for example by suitable design of the hardware and communication interfaces of enclosure 2003 itself.

Reference is now made to FIGS. 14A-14B, which schematically represent an expanded configuration of a modular robotic endoscope system 2010C, according to some embodiments of the present disclosure.

Again, most components shown are shared with the embodiments of FIGS. 12A-13C. The difference is the use of a single four-position introducer 2200, 2200A. In the sample shown, three arms are provided (e.g., in the form of steerable channel 22A). The remaining port is used for an imaging device, e.g., endoscope 2001. The ports 2020 may all be the same in size and shape, in which case the system may be configured to use any combination of camera devices and arm devices suitable to need (e.g., two of each, or three camera elements and one arm). Optionally, ports 2020 are differentiated, e.g., in embodiments for which endoscope 2001 is differently sized than steerable channels 22A.

Introducer 2200 has a rounded-corner square cross-sectional shape (which may allow a somewhat reduced cross-sectional area for the same port size), while introducer 2200A has a circular cross-sectional shape (which may be preferable, e.g., due to its radial symmetry, which means it cannot be accidentally turned in place to “expand” a tight-fitting body cavity). Optionally, the three robotic controllers 2002, 2002A are the same (and flexible in the relative orientation in which they connect to introducer 2200, 2200A). Optionally, robotic controller 2002A at least partially mirrors the other two (e.g., it has a mirrored enclosure 2003A). The symmetry of introducer 2200 may make special mirroring arrangements unnecessary, however.

Reference is now made to FIGS. 15A-15B, which schematically represent a 5-port modular robotic endoscope system 2310, according to some embodiments of the present disclosure.

In the embodiment of FIGS. 15A-15B, up five ports can be used. System 2310 is adapted to this 5-fold radial symmetry by converting its robotic controllers 2302 to use a roughly triangular-shaped enclosure, e.g., using up to ⅕th of a circular circumference, instead of up to ¼. FIG. 15B illustrates the same population of five ports 2020 as is shown in FIG. 15A—three arms (at top) and two camera elements (bottom). Again, ports 2020 are optionally all identical, but may be different. As for, e.g., FIGS. 13A-14B, other arrangements of port usage are optionally populated according to need.

Brief reference is also made to FIG. 15C, which schematically represents a port arrangement of a 3-port modular robotic endoscope system, according to some embodiments of the present disclosure. In this case, introducer 2350 is sized to provide three ports, populated, for example, with one camera element and two arms as shown. This arrangement allows a single enclosure of a robotic controller to span up to 120° of a circular circumference. Accordingly, it is potentially compatible with (e.g., optionally implemented using) the triangular enclosures 2302 of FIG. 15A, or the square aspect-ratio enclosures of FIGS. 12A, 13A, and 14A.

The remainder of the features now described in relation to FIG. 15A are optionally applied to embodiments of any of FIGS. 12A-14B.

Proximal ends 2001B of endoscopes 2001 are shown disconnected from the robotic controllers 2302, e.g., they may receive power and/or transmit their image signals through a different pathway.

Proximal-side portions of elements of steerable channel 22A are shown as hidden lines within the enclosures 2303 of robotic controllers 2302. It may be noted that proximal side 1605 of inner channel tube 1605 protrudes from proximal side 21B of middle channel tube 21, and this protrudes in turn from proximal side 20B of outer channel tube 20. This exposes access to each of these elements to the internal mechanics (not shown) of the robotic controllers 2302. The depth of enclosure 2302 may be adjusted to suit requirements for longitudinal motion. The depth shown is not to scale with the distal-side positions shown.

Also shown is passthrough port 2304. The proximal side 24B of bi-polar tool 24 is shown passing out of this port, allowing it to be manipulated manually, and/or by an another robotic controller (not shown). Optionally, proximal sides of other elements also protrude through port 2304. This may allow manual override and/or guidance of robotic controller 2302. Additionally or alternatively, robotic controller 2302 may exercise control to guide manual inputs, e.g., based on sensing of mechanical limits, programming that describes the target position, sensing of the tissue environment (e.g., imaged positions of markers), or another source of information.

Additionally or alternatively, using the passthrough port 2304, the functions of robotic controller 2302 may be distributed among a plurality of enclosures positioned along the longitudinal axis of the ports 2020. For example the most distal enclosure may handle outer channel tube 20, the next one (proximally) middle channel tube 21, and the third one inner channel tube 1605. A fourth (or other-numbered) enclosure is optionally responsible for manipulation of tool 24, and optionally reconfigurable or replaceable according to whatever tool is being used.

This approach to robotic control potentially enhances the modularity of systems built according to the descriptions of systems 2010, 2010B, and/or 2010C. In some embodiments, if one of the tubes, e.g., inner channel tube 1605, is unneeded for a particular port configuration, its corresponding enclosure is optionally omitted. Optionally, if a different design, e.g., of a middle channel tube 21 is needed (e.g., one with a different radius of curvature upon release), its own specialized controller enclosure is optionally swapped in. Controller enclosures optionally are capable of driving a plurality of different elements. They may sense which channel tube type and/or channel tube variant they are installed with (e.g., via RFID chip, contact pin sensing, or another method), and adjust their operation accordingly, if possible. Otherwise, they may report their incompatibility to operate with the current configuration.

It should be understood that any of the features described herein relating to robotic control of embodiments of FIGS. 12A-15C, are optionally provided for embodiments described in relation to FIGS. 2A-11C, insofar as they are compatible. For example, robotic control feature which are applicable to a single steerable working channel 22A (alternatively referred to herein as a manipulator arm 201A, 201B) and/or a tool positioned therein, are optionally provided to any embodiment making use of such a steerable working channel 22A/manipulator arm 201A, 201B; and/or such a tool, even if described in the context of a different introducer and/or endoscope.

### General

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A surgical manipulator arm system comprising:
an introducer, defining a lumen having a distal aperture; and
first and second manipulator arms, sized to pass through and distally out of the introducer lumen, alongside each other and along a distal longitudinal axis of the introducer;
wherein the first and second manipulator arms:
each respectively define a lumen with a distal opening, the lumen and distal opening being sized to receive a surgical tool passing therethrough,
deploy to orient their respective distal openings so that surgical tools exiting them advance towards a shared zone within which the respective surgical tool of each manipulator arm is operable; and
wherein the advance is from mutually opposing directions along the distal longitudinal axis.

2. The surgical manipulator arm system of claim 1, wherein each of the first and second manipulator arms, upon deployment distally from the introducer, comprises:
a first straight section oriented along the distal longitudinal axis of the introducer, and reaching from the introducer to a curving section;
the curving section, curved throughout its longitudinal extent to reach a deflection angle orienting the curving section's distal longitudinal axis oblique or perpendicular to the distal longitudinal axis of the introducer; and
a second straight section, oriented along the distal longitudinal axis of the curving section, and reaching from a side of the curving section opposite the first straight section to the distal opening of the manipulator arm.

3. The surgical manipulator arm system of claim 2, wherein the respective first straight sections of each of the deployed first and second manipulator arm are advanced from the introducer to different longitudinal extents, and the respective curving sections are bent to different said deflection angles.

4. The surgical manipulator arm system of claim 2, wherein:
the deployed first manipulator arm is positioned with the deflection angle of its curved section being less than 90°; and
the deployed second manipulator arm is positioned with the deflection angle of its curved section being 90° or more.

5. The surgical manipulator arm system of claim 2, wherein the curved section bends through a radius of curvature which is less than a diameter of the lumen of the introducer.

6. The surgical manipulator arm system of claim 5, wherein the deflection angle of the curved section adjusts according to how far the curved section is longitudinally advanced relative to the first straight section.

7. The surgical manipulator arm system of claim 5, wherein the curved section bends unidirectionally between the first straight section and the second straight section.

8-9. (canceled)

10. The surgical manipulator arm system of claim 5, wherein the second straight section remains in a same plane with the first straight section as the curved section bends.

11. The surgical manipulator arm system of claim 10, wherein a region of coplanarity extends from at least where the first straight section leaves the introducer, and through the curved section and second straight section up to the distal opening of the manipulator arm.

12. (canceled)

13. The surgical manipulator arm system of claim 2, wherein the first straight section, the curving section, and the second straight section comprise tubular members, defining a lumen sized for passing a surgical tool out to and beyond the distal opening of the manipulator arm.

14. The surgical manipulator arm system of claim 13, wherein at least a portion of the curving section deploys from inside the lumen of the first straight section, and at least a portion of the second straight section deploys from inside the curving section.

15. The surgical manipulator arm system of claim 13, wherein the first straight section is controllable to rotate around a longitudinal axis of the first straight section, relative to the introducer.

16. The surgical manipulator arm system of claim 13, wherein the second straight section is controllable to rotate around a longitudinal axis of the second straight section, relative to the curved section.

17. The surgical manipulator arm system of claim 1, comprising an auxiliary introducer inside the introducer, and wherein a lumen of the auxiliary introducer guides the relative positioning of the first and second manipulators as they deploy distally from the introducer.

18. The surgical manipulator arm system of claim 17, wherein the lumen of the auxiliary introducer is sized so that the first and second manipulator arms are held in position by contact both with the lumenal wall of the lumen, and with each other.

19. (canceled)

20. The surgical manipulator arm system of claim 1, wherein the manipulator arms deploy to define a shared action zone located beyond their respective distal openings, accessible by a tool deployed to extend straight beyond the distal opening of either of the manipulator arms.

21. The surgical manipulator arm system of claim 20, wherein the shared action zone is positioned longitudinally distanced from the distal end of the first straight section of the first manipulator arm by less than a diameter-length of the introducer.

22. The surgical manipulator arm system of claim 21, wherein the shared action zone is positioned radially distanced by at least one radius-length beyond a distal axial shadow of the introducer.

23. The surgical manipulator arm system of claim 1, comprising a camera module, wherein the camera module deploys to orient a field of view of at least one camera of the camera module with a central view axis pointing proximally along the distal longitudinal axis.

24. The surgical manipulator arm system of claim 23, comprising a second camera, oriented with a field of view having a central view axis pointing distally along the distal longitudinal axis.

25. The surgical manipulator arm system of claim 1, comprising a side-pointing camera module, wherein a central viewing axis of a field of view of a camera of the camera module is oriented to point within 30° of an axis perpendicular to the distal longitudinal axis of the introducer.

26. The surgical manipulator arm system of claim 25, wherein the central viewing axis of the camera oriented to point within 15° of the axis perpendicular to the distal longitudinal axis of the introducer.

27. The surgical manipulator arm system of claim 25, wherein the camera deploys to a position radially beyond a distal axial shadow of the introducer.

28-53. (canceled)
